(19) United States
(12) Patent Application Publication
(10) Pub. No.: US 2026/0020751 A1
(43) Pub. Date: Jan. 22, 2026

(54) RADIALLY EXPANDING SHEATHS

(71) Applicant: Boston Scientific Scimed, Inc., Maple Grove, MN (US)

(72) Inventors: Sean POWELL, Holden, MA (US); Gene Thomas STORBECK, Millis, MA (US)

(73) Assignee: Boston Scientific Scimed, Inc., Maple Grove, MN (US)

(21) Appl. No.: 19/270,701

(22) Filed: Jul. 16, 2025

Related U.S. Application Data
(60) Provisional application No. 63/672,366, filed on Jul. 17, 2024.

(51) Int. Cl. A61B 1/01

(52) U.S. Cl. CPC A61B 1/01

(57) ABSTRACT

A medical device may include a sheath. The sheath may have a rigid portion and a flexible portion. In a first configuration of the medical device, the sheath may include a first lumen and a second lumen. In a second configuration of the medical device, the sheath may include only the first lumen.

### CROSS-REFERENCE TO RELATED APPLICATIONS

This application claims the benefit of priority to U.S. Provisional Application No. 63/672,366, filed on Jul. 17, 2024, which is incorporated by reference herein in its entirety.

### TECHNICAL FIELD

Various aspects of the disclosure relate generally to medical devices for delivery of therapeutic agents, fluids, or medical tools. Examples of this disclosure relate to multi-lumen sheaths where regions of the sheaths are capable of expanding radially and/or moving a device or material in a radial direction.

### BACKGROUND

Medical devices used during endoscopic or other procedures, e.g., catheters, endoscopes, tubes, sheaths, and the like, require a set of properties that allow the devices to function as intended. For example, during a procedure such as an endoscopy, colonoscopy, and/or endoscopic retrograde cholangiopancreatogram (“ERCP”), an operator may deliver a material and/or insert a medical device into a patient via a lumen of a sheath. The operator may guide the sheath through tortuous anatomy for positioning the device and/or delivering the material at a target site deep in the body. Conventionally, a size of the sheath or a working channel thereof has limited a size of a material and/or device that may be delivered.

### SUMMARY

Aspects of the disclosure relate to, among other things, devices, medical devices configured for delivering therapeutic agents, fluids, and/or medical tools. Each of the aspects disclosed herein may include one or more of the features described in connection with any of the other disclosed aspects.

According to certain aspects of the disclosure, a medical device may include a sheath. The sheath may have a rigid portion and a flexible portion. In a first configuration of the sheath, the sheath may include a first lumen and a second lumen. In a second configuration of the sheath, the sheath may include only the first lumen.

Any of the devices disclosed herein may include any of the following features, additionally or alternatively, in any combination. The first lumen may be defined by a wall of the rigid portion and a wall of the flexible portion. The flexible portion may include a first arm and a second arm. The second lumen may be defined by a wall of the flexible portion, the first arm, and the second arm. In the second configuration, the first arm and the second arm may be configured to bend radially outward.

The first lumen may be configured to receive a first tool or a first material. The second lumen may be configured to receive a second tool or a second material. Upon insertion of the first tool or first material, the sheath may transition from the first configuration to the second configuration. In the second configuration, the second tool or the second material may be released from the second lumen.

The flexible portion may further include a first wall portion, a second wall portion, and a third wall portion. The first wall portion and the third wall portion may be fixed to the rigid portion. The second wall portion may extend between the first wall portion and the third wall portion. In the first configuration, the second wall portion of the flexible portion may be folded to define the second lumen. In the second configuration, the second wall portion may be unfolded to define a portion of an outer diameter of the sheath.

The rigid portion may be a first rigid portion. The flexible portion may be a first flexible portion. The sheath may include a second rigid portion and a second flexible portion. In the first and second configurations, the first rigid portion, the first flexible portion, the second rigid portion, and the second flexible portion may define an outer perimeter of the sheath. A largest width of the sheath in the first configuration may be smaller in the first configuration than in the second configuration.

The sheath may further include a third lumen in the first configuration. In the second configuration, the third lumen may merge with the second lumen. The first flexible portion may be between the first rigid portion and the second rigid portion. The second flexible portion may be between the first rigid portion and the second rigid portion, opposite of the first rigid portion. To transition the device from the first configuration to the second configuration, a tool, a material, or a fluid may be inserted through the first lumen.

The medical device may further comprise a handle at a proximal end of the sheath. The handle may be configured to provide fluid communication between the first port and the first lumen and a second port and the second lumen. The rigid portion may define a plurality of ancillary lumens.

In another example, a medical device may include a sheath. The sheath may have a rigid portion and a flexible portion fixed to the rigid portion. The flexible portion may include a first wall portion, a second wall portion, and a third wall portion. In a first configuration, the second wall portion may be folded radially inward and define a first lumen. The first wall portion, the third wall portion, and the rigid portion may define a circumference of the sheath in the first configuration. In a second configuration, the second wall portion may be unfolded. The first wall portion, the second wall portion, the third wall portion, and the rigid portion may define a circumference of the sheath.

Any of the devices disclosed herein may include any of the following features, additionally or alternatively, in any combination. The sheath may further include a second lumen. The second lumen may be defined by the rigid portion and the flexible portion in the first and second configurations. The rigid portion may define a plurality of ancillary lumens. A largest diameter of the sheath in the first configuration may be less than a largest diameter of the sheath in the second configuration.

In another example, a medical device may include a sheath having a first rigid portion, a second rigid portion, a first flexible portion, and a first lumen. The first flexible portion may be disposed between the first rigid portion and the second rigid portion. The flexible portion may include a first wall portion, a second wall portion, and a third wall portion. The first lumen may be defined by the first rigid portion, the second rigid portion, and the first flexible portion. In a first configuration, the second wall portion may be folded radially inward and define a second lumen. In a second configuration, the second wall portion may be unfolded, and the first wall portion, the second wall portion, and the third wall portion, the first rigid portion and the second rigid portion may define a circumference of the sheath.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by various aspects of the elements and combinations particularly pointed out in the appended claims.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts an exemplary medical device, according to aspects of this disclosure.

FIGS. 2A and 2B depicts a cross-section of an exemplary sheath in a first configuration (FIG. 2A) and in a second configuration (FIG. 2B), according to aspects of this disclosure.

FIGS. 3A and 3B depicts a cross-section of another exemplary sheath in a first configuration (FIG. 3A) and in a second configuration (FIG. 3B), according to aspects of this disclosure.

FIG. 4 depicts a cross-section of another exemplary sheath in a partially expanded configuration, according to aspects of this disclosure.

FIGS. 5A and 5B depict a cross-section of another exemplary sheath in a first configuration (FIG. 5A) and in a second configuration (FIG. 5B), according to aspects of this disclosure.

FIGS. 6A and 6B depict a cross-section of another exemplary sheath in a first configuration (FIG. 6A) and in a second configuration (FIG. 6B), according to aspects of this disclosure.

FIGS. 7A-7C depict a cross-section of another exemplary sheath in a first configuration (FIG. 7A), a second configuration (FIG. 7B), and a third configuration (FIG. 7C), according to aspects of this disclosure.

FIGS. 7D and 7E depict a cross-section of another exemplary sheath in a first configuration (FIG. 7D) and in a second configuration (FIG. 7E), according to aspects of this disclosure.

FIGS. 8A and 8B depict a cross-section of another exemplary sheath in a first configuration (FIG. 8A) and in a second configuration (FIG. 8B), according to aspects of this disclosure.

FIGS. 9A and 9B depict a cross-section of another exemplary sheath in a first configuration (FIG. 9A) and in a second configuration (FIG. 9B), according to aspects of this disclosure.

FIGS. 10A and 10B depict a cross-section of another exemplary sheath in a first configuration (FIG. 10A) and in a second configuration (FIG. 10B), according to aspects of this disclosure.

FIG. 11A depicts a cross-section of another exemplary sheath, and FIG. 11B depicts a perspective view of a section of the exemplary sheath of FIG. 11A with portions of the exemplary sheath removed.

FIGS. 12A and 12B depict a longitudinal cross-section of another exemplary sheath in a first configuration (FIG. 12A) and in a second configuration (FIG. 12B), according to aspects of this disclosure.

### DETAILED DESCRIPTION

Reference will now be made in detail to aspects of the disclosure, examples of which are illustrated in the accompanying drawings. Wherever possible, the same or similar reference numbers will be used through the drawings to refer to the same or like parts. Any of the devices disclosed herein may include any of the following features, additionally or alternatively, in any combination. Accordingly, between different embodiments, like numbers will be used to refer to like features, with “100” added to each numeral.

The term “distal” refers to a portion farthest away from a user when introducing a device into a subject (e.g., a patient). By contrast, the term “proximal” refers to a portion closest to the user when placing the device into the subject.

Embodiments of the disclosure may solve one or more of the limitations in the art. The scope of the disclosure, however, is defined by the attached claims and not the ability to solve a specific problem. The disclosure, in certain embodiments, is drawn to medical devices including at least one multi-lumen feature (e.g., a sheath such as a working channel) having one or more portions that are capable of expanding in a radial direction. Although the disclosure may refer at different points to one of a duodenoscope or an endoscope, it will be appreciated that, unless otherwise specified, duendoscopes, endoscopes, colonoscopes, ureteroscopes, bronchoscopes, laparoscopes, sheaths, catheters, grafts, sheaths, sheaths, any suitable delivery device or other medical device may be used in connection with aspects of this disclosure.

In aspects, an operator may desire to pass a material and/or device through a medical device (e.g., through a lumen of a sheath, such as a working channel). In some aspects, the material and/or device may be larger than the lumen of the sheath. Portions of the sheath may have varying characteristics, such as the ability to expand in a radial direction and/or release a medical device or material in a radial direction. Portions of the sheath may have one or more lumens that may expand radially. In these aspects, the sheath may include lumens or portions of lumens having greater flexibility or elasticity, for example, to permit radial expansion of the lumen and/or sheath. This may allow the one or more lumens of the sheath to receive devices and/or materials (e.g., boluses) that are larger than the lumens or channels of the sheath when the lumens or channels are in a contracted configuration.

Flexible portions and rigid portions of sheaths are described herein. Unless otherwise stated, it may be understood that the described “rigid” portions may less flexible than the described “flexible” portions, but may still exhibit characteristics of a flexible material. In other words, the “rigid” portions may be flexible so as to bend and/or flex. However, the rigid portions may be less flexible as compared to the flexible portions. Furthermore, the flexible portions described herein may be configured to flex, bend, and/or stretch. It may also be understood that the flexible portions and rigid portions described herein may or may not be comprised of different materials. For example, the flexible portions and the rigid portions may be comprised of a same material.

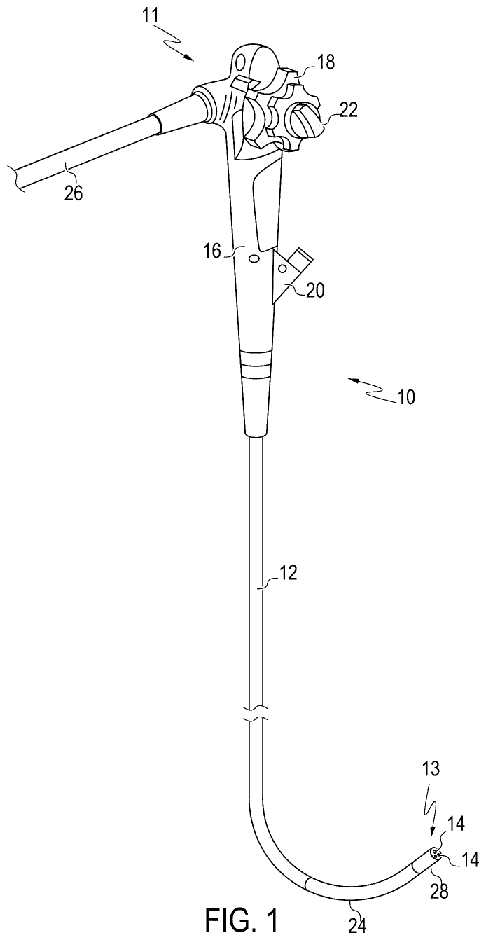

FIG. 1 illustrates an exemplary medical device 10 (e.g. an endoscope) in accordance with an embodiment of this disclosure. Medical device 10 includes a proximal end 11 and a distal end 13. A handle 16, with one or more knobs 18, locks 22, and/or ports 20, is at proximal end 11. A shaft 12 extends from a distal end of handle 16 to a distal end 13 of device 10.

Shaft 12 is a sheath or tube having sufficient length and flexibility to access sites within the body and traverse tortuous anatomy. Shaft 12 may be a single component with one or more lumens 14 extending through a longitudinal length of shaft 12. In other aspects, lumen(s) 14 of shaft 12 may be comprised of multiple sheaths or multiple components. For example, shaft 12 may be comprised of multiple sheaths (e.g., working channels) having single or multiple lumens that is/are contained within a lumen of a larger sheath.

Lumen(s) 14 may comprise any number of lumens desired and as permitted given the constraints associated with the cross sectional area and desired characteristics of shaft 12. For example, lumens 14 may include lumens for insertion/delivery of instruments, delivery of fluid (e.g., treatment or prophylactic agents, air, and/or water), application of suction, housing of electrical cables or wires, housing of wires for steering (connecting the handle to a distal portion), etc. One or more lumens 14 may open at a distal face of shaft 12, as shown in FIG. 1, for example, to deliver fluid or an instrument distal to the end of device 10. Additionally or alternatively, one or more lumens 14 may terminate at a position proximal to the distal face of shaft 12. In some aspects, a cap 28 may be placed at a distal end of shaft 12 to close the end of one or more of the lumen(s) 14, for example, to enclose electrical cabling for imaging and lighting and/or to direct the opening of one or more lumens 14 in one or more directions.

Two or more lumens 14 may be substantially parallel to each other, so that longitudinal axes of the two or more lumens 14 are substantially parallel to one another and to the longitudinal axis of the shaft 12. Lumen(s) 14 may comprise a variety of shapes (e.g. square, oval, circular, star, etc.) and sizes. In some aspects, the shapes and/or sizes of lumen(s) 14 may change. For example, a size and/or shape of lumen(s) 14 may be different in a first (unexpanded or contracted) configuration as compared to a second (expanded or enlarged) configuration.

Shaft 12 may be connected to handle 16 at a proximal end of shaft 12, thus permitting access to one or more of the plurality of lumens 14 from the one or more ports 20. Additionally or alternatively, a proximal end of one or more lumen(s) 14 may terminate inside handle 16. In some aspects, one or more lumen(s) 14 may communicate with structures within the handle and/or an umbilicus 26. Such structures may permit suction, insufflation, irrigation, and/or the connection of electronics, etc., as is known in the art.

Medical device 10 may further include an articulation portion 24 for navigating tortuous anatomy and directing the distalmost end of device 10 towards a target site. Articulation portion 24 may be located adjacent to or just proximal of distal end 13 of medical device 10. For example, articulation portion 24 may be fixed to or integrally formed with a distal end of shaft 12. Articulation portion 24 may enable a user to bend or articulate distal end 13 in any desired direction (e.g., up, down, left, and/or right) through known means in the art, particularly through the manipulation of knobs 18 and control wires contained within the handle 16 and lumen(s) 14 of shaft 12. Cap 28 may be coupled to a distalmost end of articulation portion 24.

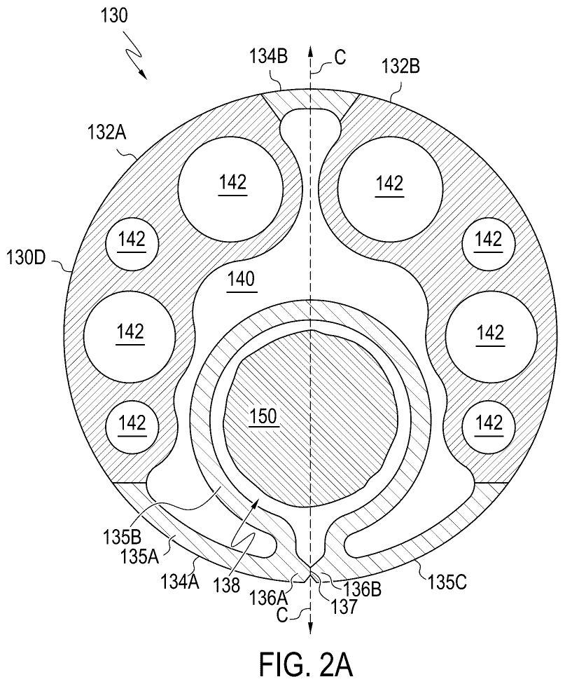

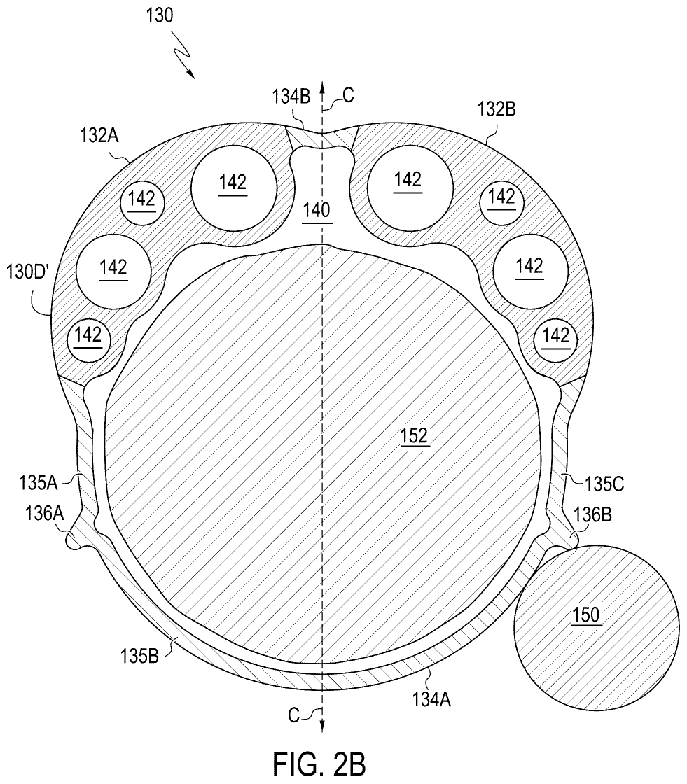

FIG. 2A illustrates cross-sectional view of an exemplary sheath 130 in a first, contracted configuration. FIG. 2B illustrates sheath 130 in a second, expanded configuration. Sheath 130 may be used as shaft 12 of FIG. 1. Alternatively, sheath 130 may extend through shaft 12 of FIG. 1, thus operating as a working channel or other sheath extending through shaft 12 (e.g., as one or more lumens 14). In these aspects, one or more lumens of sheath 130 may be in fluid communication with one or more ports 20 of handle 16 of FIG. 1).

In aspects, sheath 130 may be symmetric about a centerline “C” that may extend perpendicularly to a longitudinal axis of sheath 130. The longitudinal axis of sheath 130 may extend into and out of the page in FIGS. 2A and 2B. Accordingly, features of sheath 130 on one side (e.g., the left or right side) of centerline “C” may also be on the other side (e.g., the right or left side) of centerline “C,” as shown in FIGS. 2A and 2B. In other aspects, sheath 130 may be asymmetrical. For example, features of sheath 130 on one side (e.g., the left or right side) of centerline “C” may not be on the other side (e.g., the right or left side) of centerline “C”, or vice versa.

Sheath 130 may include one or more rigid portions and one or more flexible portions. The rigid portions and flexible portions may extend from a proximal end to a distal end of sheath 130. In examples, sheath 130 may have a same cross-sectional arrangement along an entirety or a portion of sheath 130. In other words, the rigid portions and/or the flexible portions may be uniform along an entire length or a partial length of sheath 130. For example, sheath 130 may include a first rigid portion 132A and a second rigid portion 132B (e.g., on an opposite side of centerline “C”). Sheath 130 may also include a first flexible portion 134A and a second flexible portion 134B, each extending between first rigid portion 132A and second rigid portion 132B. In aspects, first flexible portion 134A and second flexible portion 134B may extend across centerline “C,” for example, between rigid portions 132A, 132B.

Rigid portion(s) 132A, 132B and flexible portion(s) 134A, 134B may be integrally formed or otherwise fixedly coupled to one another (e.g., overmolded, co-extruded, or otherwise manufactured to form a single piece). For example, in the first and second configurations (e.g., FIGS. 2A and 2B), outer surfaces of rigid portions 132A, 132B and flexible portions 134A, 134B may form a continuous outer wall 130D of sheath 130. As will be explained in more detail below, the portions of flexible portion 134A forming continuous outer wall 130D′ of sheath 130 may differ in the second configuration (FIG. 2B).

The material(s) comprising rigid portions 132A, 132B and/or flexible portions 134A, 134B may include one or more of a polyvinylchloride (PVC), high density polyethylene (HDPE), polyurethane, nylon, PEBAX®, fluorinated ethylene propylene, perfluoroalkoxy, ethylene tetrafluoroethylene (ETFE), polyetheretherketone (PEEK), thermoplastic elastomer (TPE), polytetrafluoroethylene (PTFE), expanded polytetrafluoroethylene (ePTFE), and/or any other material commonly used to manufacture medical sheaths. In examples, rigid portion(s) 132A, 132B and flexible portion(s) 134A, 134B may be formed of a same material, yet have different durometers. In other aspects, rigid portion(s) 132A, 132B and flexible portion(s) 134A, 134B may each be formed of different materials having different durometers. In some aspects, flexible portion(s) 134A, 134B may include materials having shape-memory capabilities. For example, when flexible portion(s) 134A, 134B is/are unfolded or otherwise deformed, flexible portion(s) 134A, 134B may return to a pre-deformed shape.

Referring to FIG. 2A, first flexible portion 134A may include a first wall portion 135A, a second wall portion 135B, and a third wall portion 135C. For example, a first end of first wall portion 135A may be fixed to, extend from, and/or be integrally formed with first rigid portion 132A, and a first end of third wall portion 135C may be fixed to, extend from, and/or be integrally formed with second rigid portion 132B. Second wall portion 135B may extend between a second end of first wall portion 135A and a second end of second wall portion 135B.

In aspects, a first arm 136A may be disposed between first wall portion 135A and second wall portion 135B (e.g., at a junction of first wall portion 135A and second wall portion 135B), and a second arm 136B may be disposed between second wall portion 135B and third wall portion 135C (e.g., at a junction of second wall portion 135B and third wall portion 135C). In the first configuration (FIG. 2A), first wall portion 135A, first arm 136A, third wall portion 135C, and second arm 136B may form a continuous wall and, thus, form a portion of an outer wall 130D of sheath 130. For example, in the first configuration (FIG. 2A), an end of each of first arm 136A and second arm 136B may meet at a point 137. In some aspects, the ends of first arm 136A and second arm 136B may abut one another at point 137. In other aspects, first arm 136A and second arm 136B may be coupled at point 137, for example, via an adhesive or other bonding substance. In yet further aspects, the ends of first arm 136A and second arm 136B may be coupled or fused together (e.g., integrally formed with one another) at point 137.

Sheath 130 may include perforations, for example, at point 137. The perforations may extend longitudinally along a length of sheath 130 such that, for example, when sheath 130 transitions to sheath 130 of FIG. 2B, the perforations break at point 137. Additionally or alternatively, a drawstring or rip-cord embedded within a wall of sheath 130, for example, at point 137, may be used to facilitate separation of first arm 136A from second arm 136B. In other aspects, first arm 136A may otherwise be decoupled from second arm 136B, for example, when sheath 130 transitions to the second configuration.

With first arm 136A and second arm 136B abutting one another, or otherwise coupled together, e.g., at point 137, at least a portion (e.g., second wall portion 135B) of first flexible portion 134A may be folded to form a first central lumen 138 in the first configuration (FIG. 2A). For example, in the first configuration, first central lumen 138 may be defined by second wall portion 135B, first arm 136A, and second arm 136B. For example, in the first configuration, second wall portion 135B may be curved to form a portion of a circumference of a circle. First central lumen 138 may be configured to receive a first tool 150. First tool 150 may be a guide wire, a basket, a snare, forceps, or any other medical tool commonly used in the art.

Sheath 130 may include a second central lumen 140 that at least partially surrounds first central lumen 138 in the first configuration (FIG. 2A). For example, second central lumen 140 may be defined by rigid portions 132A, 132B, and flexible portions 134A, 134B. First central lumen 138 and second central lumen 140 may not be in fluid communication. For example, first central lumen 138 may be isolated from second central lumen 140 in the first configuration (FIG. 2A), for example, by second wall portion 135B of first flexible portion 134A.

In the second (expanded) configuration, second central lumen 140 may be configured to receive a second tool 152. Second tool 152 may be a guide wire, a basket, a snare, forceps, or any other medical tool commonly used in the art. Second tool 152 may have a diameter larger than a diameter of second central lumen 140. For example, when second central lumen 140 receives second tool 152, sheath 130 may expand. When second central lumen 140 receives second tool 152, first arm 136A and second arm 136B may spread (e.g., break) apart, thus opening first central lumen 138 to an environment surrounding sheath 130. For example, when second tool 152 is received within second central lumen 140, first arm 136A and second arm 136B may break, or split, apart or no longer abut one another at point 137 and second wall portion 135B of first flexible portion 134A may unfold. In the second configuration, first arm 136A and second arm 136B may protrude radially outward, for example, away from the central longitudinal axis of sheath 130.

Second central lumen 140 may continue to expand, for example, until first tool 150 is released from first central lumen 138. For example, in the second configuration (FIG. 2B) second wall portion 135B of first flexible portion 134A may form a continuous, convex wall and, thus, form a portion of an expanded outer wall 130D′ of sheath 130, along with first wall portion 135A and third wall portion 135C. In aspects, first tool 150 may be released radially (e.g., from a radial side of sheath 130).

In the second configuration (FIG. 2B), rigid portions 132A, 132B may bow outwards (e.g., away from centerline “C”) via second flexible portion 134B. For example, second flexible portion 134B may be in a concave position in the first configuration (FIG. 2A). In the second configuration (FIG. 2B), second flexible portion 134B may be in a convex position, thus biasing rigid portions 132A, 132B away from centerline “C.” In these aspects, second flexible portion 134B may act as a hinge.

In aspects, a cross-sectional area of second central lumen 140 may increase in the second configuration. Furthermore, an overall diameter/width of sheath 130 may increase from the first configuration to the second configuration. For example, a largest diameter/width of sheath 130 may be less than, or smaller, in the first configuration than in the second configuration. In the first configuration, sheath 130 may have an approximately circular outer perimeter. In the second configuration, sheath 130 may have a non-circular outer perimeter. Sheath 130 may transition from the second configuration (FIG. 2B) back to the first configuration (FIG. 2A), for example, by removing second tool 152 from second central lumen 140. When second tool 152 is removed from second central lumen 140, second wall portion 135B may fold radially inward, for example, re-forming first central lumen 138. Sheath 130 may be biased to the first configuration. Additional tools may be inserted through first central lumen 138 and/or second central lumen 140, for example, throughout a procedure.

In some aspects, sheath 130 may transition from the first configuration (FIG. 2A) to the second configuration (FIG. 2B) only if first tool 150 of a sufficient size is passed through first central lumen 138 and/or only if second tool 152 of a sufficient size is passed through second central lumen 140. For example, if second tool 152 is not larger than second central lumen 140 (e.g., when sheath 130 is in the first configuration), sheath 130 may not transition from the first configuration to the second configuration. In other examples, inserting a first tool 150 that is larger than first central lumen 138, may transition sheath 130 to the second configuration (FIG. 2B), for example, with or without inserting second tool 152 through second central lumen 140.

In other aspects, sheath 130 may transition from the first configuration to the second configuration by injecting a fluid (e.g., a gas or a liquid) into second central lumen 140. The fluid may be a bolus. For example, second central lumen 140 may be sealed or closed off at a distal end such that the fluid is contained within second central lumen 140. As a volume of the fluid increases within second central lumen 140 (e.g., thus increasing a pressure within second central lumen 140), the fluid may force sheath 130 into the second configuration (FIG. 2B). Similarly, when the fluid is removed from second central lumen 140, sheath 130 may transition back to the first configuration (FIG. 2A).

It may also be understood that sheath 130 may transition from the first configuration to the second configuration, for example, if a material or fluid is being removed from the subject through first central lumen 138 and/or second central lumen 140. For example, if a material (e.g., a stone or foreign object) is removed from the subject via second central lumen 140 that is larger than a diameter of second central lumen 140, sheath 130 may transition to the second configuration (FIG. 2B). In these aspects, materials that are larger than a diameter of the second central lumen 140 may be removed from the subject.

In some aspects, sheath 130 may transition from the first configuration (FIG. 2A) to the second configuration (FIG. 2B) only if first tool 150 of a sufficient size is passed through first central lumen 138 and/or only if second tool 152 of a sufficient size is passed through second central lumen 140. For example, if second tool 152 is not larger than second central lumen 140 (e.g., in the first configuration), sheath 130 may not transition from the first configuration to the second configuration.

Still referring to FIGS. 2A and 2B, one or more ancillary lumens 142 may extend through each of rigid portion(s) 132A, 132B of sheath 130. Ancillary lumen(s) 142 may be configured to receive components (e.g., one or more articulation wires, electrical components such as electrical wires or cables, etc.). Additionally or alternatively, ancillary lumen(s) 142 may be configured to receive a fluid and/or a material. Each of ancillary lumen(s) 142 may have a same diameter and/or a same shape. In other aspects, one or more of ancillary lumen(s) 142 may have different diameters and/or different shapes. The size and shape of ancillary lumen(s) 142 may remain unchanged from the first configuration to the second configuration.

As shown in FIGS. 2A and 2B, four ancillary lumens 142 may extend through each of rigid portions 132A, 132B. In further aspects, ancillary lumens 142 may be omitted from one or both of rigid portions 132A, 132B. For example, sheath 130 may only include first central lumen 138 and second central lumen 140 in the first configuration and second central lumen 140 in the second configuration. Omitting ancillary lumen(s) 142 may allow for a larger cross-sectional area of first central lumen 138 and/or second central lumen 140 in the first configuration and/or of second central lumen 140 in the second configuration (e.g., due to a smaller size of rigid portions 132A and/or 132B).

In some aspects, a material may be delivered via any of the lumens of sheath 130 (e.g., via first central lumen 138, second central lumen 140, and/or ancillary lumens 142). For example, a first material may be delivered via first central lumen 138. A second material may be delivered via second central lumen 140. Together or separately, each of the first material and second material may be injected through their respective lumens of sheath 130 and delivered to the target site. Once the materials come in contact with one another (e.g., after being delivered from their respective lumens), the materials may crosslink. The cross-linked structure of the resulting combination of the materials may have enhanced hemostatic properties compared the fluids individually. In a non-limiting example, the first material may include fibrinogen (e.g., lyophilized pooled human concentrate) and the second material may include thrombin (e.g., of bovine or human origin). The biocompatible fibrinogen and thrombin mixture may also contain calcium salts. In aspects, a third material may be delivered via the one or more ancillary lumen 142.

In further aspects, the one or more ancillary lumen 142 may be omitted from sheath 130. For example, sheath 130 may only include first central lumen 138 and second central lumen 140 in the first configuration. In the second configuration, sheath 130 may only include second central lumen 140.

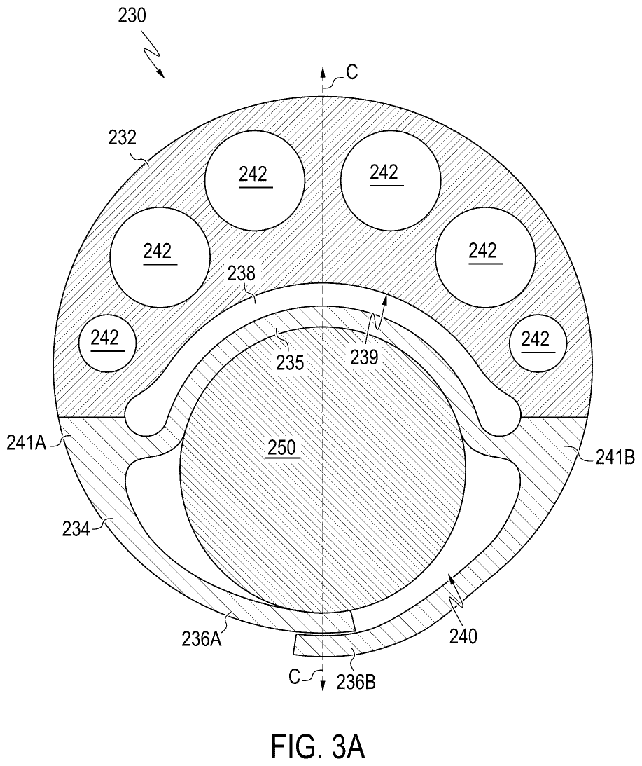

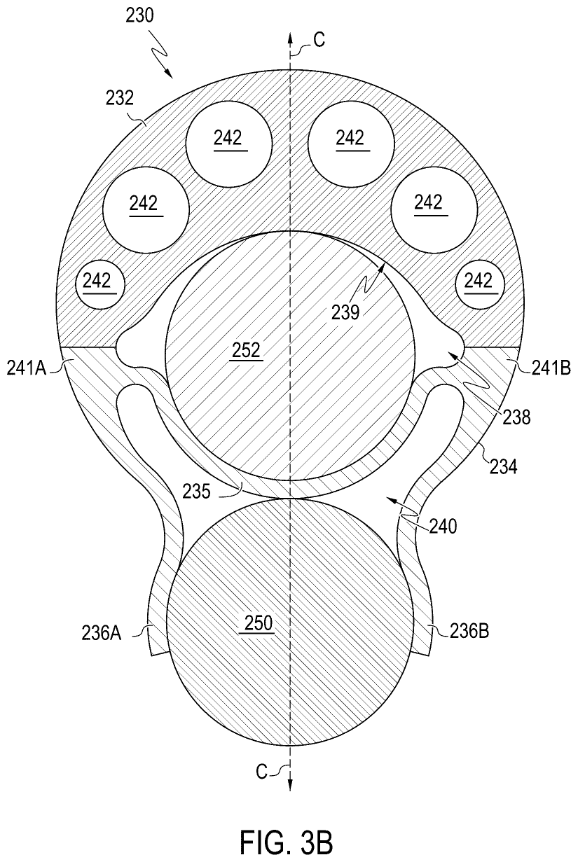

FIGS. 3A and 3B illustrate cross-sectional view of another exemplary sheath 230 in a first configuration (FIG. 3A) and a second configuration (FIG. 3B). Sheath 230 may have any or all of the same characteristics of sheath 130, described above with respect to FIGS. 2A and 2B, except as described below. For example, sheath 230 may include a rigid portion 232 and a flexible portion 234. In aspects, sheath 230 may be symmetric about a centerline “C” that may extend perpendicularly to a longitudinal axis of sheath 230. The longitudinal axis of sheath 230 may extend into and out of the page in FIGS. 2A and 2B. In other aspects, sheath 230 may be asymmetric. For example, features of sheath 230 on one side (e.g., the left or right side) of centerline “C” may not be on the other, opposite side (e.g., the right or left side) of centerline “C”, or vice versa.

Rigid portion 232 may have any or all of the same characteristics of one or both of rigid portions 132A, 132B of FIGS. 2A and 2B. Flexible portion 234 may have any or all of the same characteristics of one or both of flexible portions 134A, 134B of FIGS. 2A and 2B. In some aspects, rigid portion 232 and flexible portion 234 may be integrally formed or otherwise fixedly coupled to one another (e.g., overmolded or co-extruded).

Flexible portion 234 may include a wall portion 235. Wall portion 235 may extend across centerline “C,” for example, between a first base 241A and a second base 241B of flexible portion 234. In aspects, wall portion 235 may at least partially define a first central lumen 238. First central lumen 238 may also be defined by an inner wall 239 of rigid portion 232. For example, inner wall 239 of rigid portion 232 and wall portion 235 of flexible portion 234 may together define an entire circumference of first central lumen 238. In the first configuration (FIG. 3A), first central lumen 238 may be in a collapsed position. For example, a portion of wall portion 235 of flexible portion 234 may abut or be adjacent to inner wall 239. In aspects, wall portion 235 may be rounded towards inner wall 239. In these aspects, first central lumen 238 may have a first cross-sectional area, or a first volume in the first configuration.

Flexible portion 234 may further include a first arm 236A and a second arm 236B. First arm 236A may extend from first base 241A, for example, towards and across centerline “C.” Second arm 236B may extend from second base 241B, for example, towards and across centerline “C.” In some aspects, a first end of first arm 236A may overlap with a second end of second arm 236B in the first configuration. In other aspects, first arm 236A and second arm 236B may not overlap. For example, a gap may be formed between the first end of first arm 236A and the second end of second arm 236B in the first configuration. Alternatively, the first end of first arm 236A and the second end of second arm 236B may abut, but not overlap, in the first configuration.

In some aspects, wall portion 235 may include arms that are similar to first arm 236A and second arm 236B. In other aspects, arms 236A, 236B may be replaced with features of sheath 130 (of FIGS. 2A and 2B). For example, flexible portion 234 may include a first wall portion (e.g., similar to first wall portion 135A of FIGS. 2A and 2B), a second wall portion (e.g., similar to second wall portion 135B of FIGS. 2A and 2B), and a third wall portion (e.g., similar to third wall portion 135C of FIGS. 2A and 2B) in addition to or in place of arms 236A, 236B of FIGS. 3A and 3B.

In the first configuration (FIG. 3A), first arm 236A, second arm 236B, and wall portion 235 may together define a circumference/perimeter of a second central lumen 240. Second central lumen 240 may be adjacent to first central lumen 238, separated from one another by wall portion 235. Second central lumen 240 may be configured to receive a first tool 250.

In the second configuration (FIG. 3B), a second tool 252 may be inserted through first central lumen 238. With second tool 252 inserted through first central lumen 238, wall portion 235 may be forced radially outward, such that, for example, wall portion 235 is rounded away from inner wall 239. In these aspects, first central lumen 238 may expand to receive second tool 252.

When second tool 252 is inserted through first central lumen 238, first tool 250 may be forced radially outward. As first tool 250 is forced radially outward, first arm 236A and second arm 236B may flex, or bend, outwards (e.g., away from centerline “C”). In some aspects, first arm 236A and second arm 236B may bend radially outward. An opening may be formed between first arm 236A and second arm 236B that has a width that is greater than or equal to a width/diameter of first tool 250. In these aspects, first tool 250 may be released from second central lumen 240. First tool 250 may be released radially (e.g., from a radial side of sheath 230).

Additional tools may be released from second central lumen 240. For example, first tool 250 may be removed from first central lumen 238 and a third tool (not shown) may be inserted through second central lumen 240. The third tool (not shown) may be delivered by re-inserting first tool 250 (or a fourth tool) through first central lumen 238, thus forcing the third tool radially outward and between first arm 236A and second arm 236B.

Still referring to FIGS. 3A and 3B, one or more ancillary lumens 242 may extend through rigid portion 232. Ancillary lumen(s) 242 may have any or all of the characteristics of ancillary lumens 142 of sheath 130 of FIGS. 2A and 2B. Ancillary lumen(s) 242 may be configured to receive components (e.g., one or more articulation wires, electrical components such as electrical wires or cables, etc.). Additionally or alternatively, ancillary lumen(s) 242 may be configured to receive a fluid and/or a material. Each of ancillary lumen(s) 242 may have a same diameter and/or a same shape. In other aspects, one or more of ancillary lumen(s) 242 may have different diameters and/or different shapes. For example, as shown in FIGS. 3A and 3B, six ancillary lumens 242 may extend through rigid portion 232. In further aspects, ancillary lumens 242 may be omitted from rigid portion 232.

In some aspects, a material may be delivered via any of the lumens of sheath 230 (e.g., via first central lumen 238, second central lumen 240, and/or one or more ancillary lumen(s) 242). For example, a first material may be delivered via first central lumen 238. A second material may be delivered via second central lumen 240. Together or separately, each of the first material and second material may be injected through their respective lumens of sheath 230. In aspects, a third material may be delivered via the one or more ancillary lumen(s) 242.

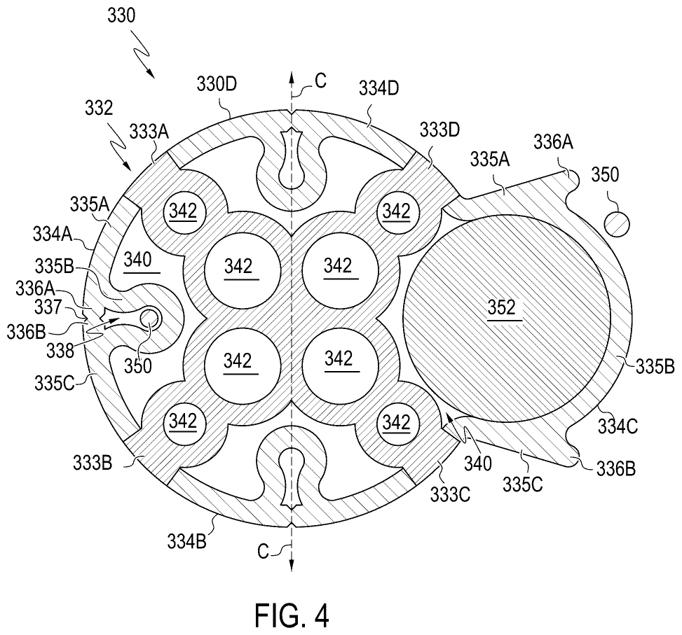

FIG. 4 illustrates a cross-section view of an exemplary sheath 330 in a partially expanded configuration. Sheath 330 may have any or all of the same characteristics of sheath 130 of FIGS. 2A and 2B and/or of sheath 230 of FIGS. 3A and 3B, except as described below. For example, sheath 330 may include a rigid portion 332, a first flexible portion 334A, a second flexible portion 334B, a third flexible portion 334C, and a fourth flexible portion 334D. In aspects, rigid portion 332 may have an “X” or cross shape with a plurality of arms extending radially outward from a longitudinal axis of sheath 330. The longitudinal axis of sheath 330 may extend into and out of the page. For example, rigid portion 332 may include a first arm 333A, a second arm 333B, a third arm 333C, and a fourth arm 333D. In aspects, flexible portions 334A, 334B, 334C, 334D may extend between two arms of rigid portion 332.

In aspects, in a fully collapsed or a fully expanded configuration, sheath 330 may be symmetric about a centerline “C” that may extend perpendicularly relative to the longitudinal axis of sheath 330. Features of sheath 330 may be arranged, for example, in a radial pattern. In other aspects, sheath 330 may be asymmetrical. For example, features of sheath 330 on one side (e.g., the left or right side) of centerline “C” may not be on the other side (e.g., the right or left side) of centerline “C”, or vice versa.

In aspects, in fully collapsed or fully contracted configurations, sheath 330 may be symmetric about multiple centerlines. For example, sheath 330 may be symmetric about two or four centerlines. In an example, sheath 330 may be symmetric about centerlines that extend through (1) centers of flexible portions 334B and 334D (centerline “C”), (2) centers of flexible portions 334A and 334C, (3) centers of arms 333A and 333C, and/or (4) centers of arms 333B and 333D.

Rigid portion 332 may have any or all of the same characteristics of rigid portions 132A, 132B of FIGS. 2A and 2B and/or rigid portion 232 of FIGS. 3A and 3B. Flexible portions 334A, 334B, 334C, and/or 334D may have any or all of the same characteristics of flexible portions 134A, 134B of FIGS. 2A and 2B and/or flexible portion 234 of FIGS. 3A and 3B. In some aspects, rigid portion 332 may be integrally formed with or otherwise fixedly coupled to flexible portions 334A, 334B, 334C, and/or 334D.

Flexible portions 334A, 334B, 334C, and 334D may have any or all of the same characteristics as one another. For ease of description, first flexible portion 334A and third flexible portion 334C are described in detail below. In these aspects, element numbers for features of first flexible portion 334A may be used to identify similar element numbers for features of third flexible portion 334C. For example, first flexible portion 334A is described herein as being in a first, or collapsed, configuration, and third flexible portion 334C is described herein as being in a second, or expanded, configuration. Second flexible portion 334B and fourth flexible portion 334D may have any or all of the same characteristics of first flexible portion 334A and third flexible portion 334C.

First flexible portion 334A and fourth flexible portion 334D may be similarly shaped as first flexible portion 134A of FIGS. 2A and 2B (and vice versa). For example, first flexible portion 334A and fourth flexible portion 334D may each have a first wall portion 335A, a second wall portion 335B, and a third wall portion 335C. First wall portion 335A may extend from a first arm 333A of rigid portion 332. Third wall portion 335C may extend from a second arm 333B of rigid portion 332. Second wall portion 335B may extend between first wall portion 335A and second wall portion 335B.

A first arm 336A may be disposed between first wall portion 335A and second wall portion 335B, and a second arm 336B may be disposed between second wall portion 335B and third wall portion 335C. In a folded configuration, first wall portion 335A, first arm 336A, third wall portion 335C, and second arm 336B may form a continuous wall and, thus, form a portion of an outer wall 330D of sheath 330. First arm 336A and second arm 336B may meet, or abut, at a point 337.

With first arm 336A and second arm 336B abutting one another, or otherwise coupled together, e.g., at point 337, at least a portion of first flexible portion 334A/fourth flexible portion 334D (e.g., second wall portion 335B) may be folded radially inward to form a first central lumen 338. For example, first central lumen 338 may be defined by second wall portion 335B, first arm 336A, and second arm 336B. First central lumen 338 may be configured to receive a first tool 350, as shown for first flexible portion 334A.

A second central lumen 340 may be formed at least partially around first central lumen 338 in a closed, or collapsed, configuration. For example, second central lumen 340 may be defined by two arms of rigid portion 332 and flexible portion 334A. In these aspects, a portion of a wall forming each rigid portion 332 and first flexible portion 334A may form a portion of a perimeter of second central lumen 340. First central lumen 338 and second central lumen 340 may not be in fluid communication. For example, first central lumen 338 may be isolated from second central lumen 340 via second wall portion 335B.

Second central lumen 340 may be configured to receive a second tool 352 and/or a material, as shown for fourth flexible portion 334D. For example, when second central lumen 340 receives first tool 350, first arm 336A and second arm 336B may break apart, thus opening first central lumen 338, as shown for fourth flexible portion 334D. For example, when second tool 352 is received within second central lumen 340, second central lumen 340 may expand radially outward. As second central lumen 340 expands radially outward to receive second tool 352, first arm 336A and second arm 336B may break or no longer abut one another at point 337 and second wall portion 335B of first flexible portion 334A may unfold. Second central lumen 340 may continue to expand, for example, until first tool 350 is released from first central lumen 338. First tool 350 may be released radially (e.g., from a radial side of sheath 330).

In some aspects, second wall portion 335B may include arms that are similar to arms 236A, 236B of FIGS. 3A and 3B. Additionally or alternatively, sheath 330 may include one or more flexible walls (e.g., similar to wall portion 235 of FIGS. 3A and 3B) extending between adjacent arms of rigid portion 332. The flexible wall(s) may divide second central lumen 340 into additional lumens (e.g., two or more lumens).

Still referring to FIG. 4, one or more ancillary lumens 342 may extend through rigid portion 332. Ancillary lumen(s) 342 may have any or all of the same characteristics of ancillary lumen(s) 142 of FIGS. 2A and 2B and/or ancillary lumen(s) 242 of FIGS. 3A and 3B. In further aspects, ancillary lumens 342 may be omitted from rigid portion 332, such that arms 333A, 333B, 333C, 333D may be substantially straight and/or may form an overall X-shape with a thin wall thickness of arms 333A, 333B, 333C, 3333D. Omitting ancillary lumen(s) 342 may allow for a larger cross-sectional area of first central lumen 338 and/or second central lumen 340.

In some aspects, a material may be delivered via any of the lumens of sheath 330 (e.g., via first central lumen 338, second central lumen 340, and/or ancillary lumen(s) 342). For example, a first material may be delivered via first central lumen 338. A second material may be delivered via second central lumen 340. Together or separately, each of the first material and second material may be injected through their respective lumens of sheath 330. In aspects, a third material may be delivered via the one or more ancillary lumen(s) 342.

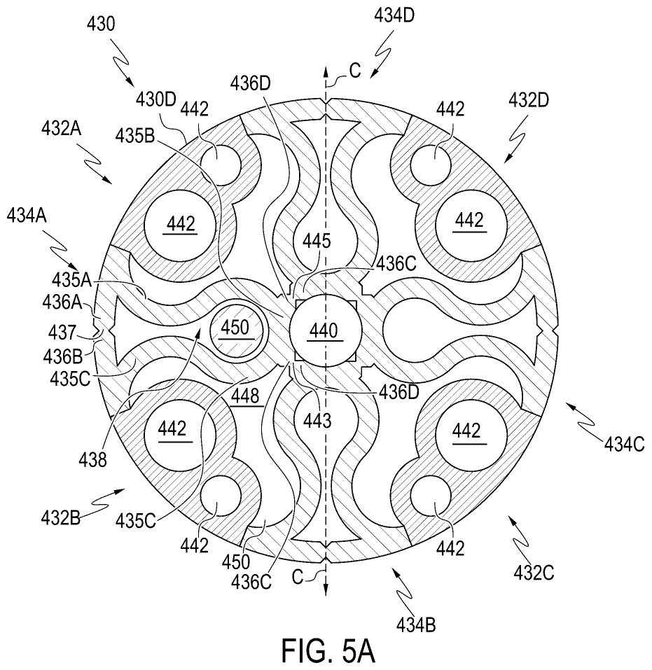

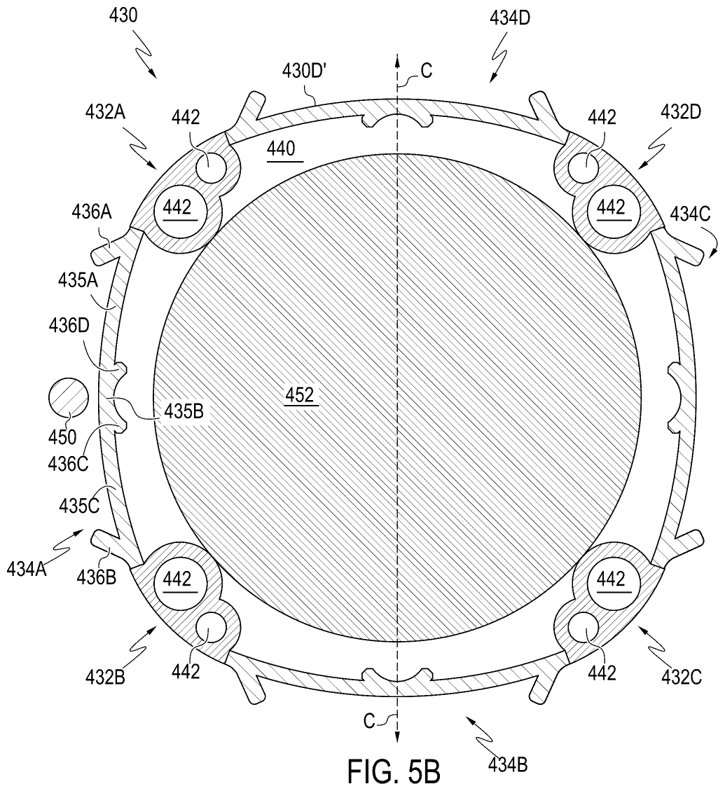

FIGS. 5A and 5B illustrate cross-sectional views of an exemplary sheath 430 in a first configuration (FIG. 5A) and a second configuration (FIG. 5B). Except as described below, sheath 430 may have any or all of the same characteristics of sheaths 130, 230, and/or 330, described above. For example, sheath 430 may include a plurality of rigid portions and a plurality of flexible portions. In particular, sheath 430 may include a first rigid portion 432A, a second rigid portion 432B, a third rigid portion 432C, and a fourth rigid portion 432D. Sheath 430 may further include a first flexible portion 434A, a second flexible portion 434B, a third flexible portion 434C, and a fourth flexible portion 434D. The rigid portions and flexible portions may be arranged radially about a central longitudinal axis (e.g., extending into and out of the page) of sheath 430, for example, in an alternating pattern. Flexible portions 434A, 434B, 434C, 434D may each have the same features or characteristics as one another. Similarly, rigid portions 432A, 432B, 432C, 432D may each have the same features or characteristics as one another. In these aspects, only elements of first flexible portion 434A and first rigid portion 432A are identified in FIGS. 5A and 5B.

In aspects, sheath 430 may be symmetric about a centerline “C” that may extend perpendicularly to the longitudinal axis of sheath 430. Features of sheath 430 may be arranged, for example, in a radial pattern. In other aspects, sheath 430 may be asymmetrical. For example, features of sheath 430 on one side (e.g., the left or right side) of centerline “C” may not be on the other side (e.g., the right or left side) of centerline “C”, or vice versa. In aspects, sheath 430 may be symmetric about multiple centerlines. For example, sheath 430 may be symmetric about two centerlines. In an example, sheath 430 may be symmetric about centerlines that extend through (1) centers of flexible portions 434B and 434D (centerline “C”) and/or (2) centers of flexible portions 434A and 434C.

First flexible portion 434A may be similarly shaped as first flexible portion 134A of FIGS. 2A and 2B and/or first flexible portion 334A of FIG. 4. For example, first flexible portion 434A may have a first wall portion 435A, a second wall portion 435B, and a third wall portion 435C. First wall portion 435A may extend from first rigid portion 432A. Third wall portion 435C may extend from second rigid portion 432B. Second wall portion 435B may extend between first wall portion 435A and third wall portion 435C.

A first arm 436A may be disposed between first wall portion 435A and second wall portion 435B, and a second arm 436B may be disposed between second wall portion 435B and third wall portion 435C. In a folded configuration, first wall portion 435A, first arm 436A, third wall portion 435C, and second arm 436B may form a continuous wall and, thus, form a portion of an outer wall 430D of sheath 430. First arm 436A and second arm 436B may meet at a point 437.

With first arm 436A and second arm 436B abutting one another, or otherwise coupled together, e.g., at point 437, at least a portion (e.g., second wall portion 435B) of first flexible portion 434A may be folded to form a first central lumen 438. For example, first central lumen 438 may be defined by second wall portion 435B, first arm 436A, and second arm 436B. First central lumen 438 may be configured to receive a first tool 450.

A third arm 436C and a fourth arm 436D may extend radially inward (e.g., towards the central longitudinal axis of sheath 430) from second wall portion 435B. Third arm 436C may meet a fourth arm 436D of an adjacent flexible portion (e.g., second flexible portion 434B) at a point 443. Similarly, fourth arm 436D may meet at a third arm 436C of an adjacent flexible portion (e.g., fourth flexible portion 434D) at a point 445. Third arm 436C and fourth arm 436D of each flexible portion 434A, 434B, 434C, 434D may together form a second central lumen 440 in a closed, or collapsed, configuration (FIG. 5A). First central lumen 438 and second central lumen 440 may not be in fluid communication. For example, first central lumen 438 may be isolated from second central lumen 440 via second wall portion 435B.

In the first configuration, third central lumens 448 may also be formed between each of two adjacent flexible portions. For example, a circumference of third central lumen 448 may be defined by third wall portion 435C of first flexible portion 434A, a first wall portion 435A of an adjacent flexible portion (e.g., second flexible portion 434B), and first rigid portion 432A. In some aspects, fourth arm 436D of first flexible portion 434A and a third arm of an adjacent flexible portion (e.g., second flexible portion 434B) may also form a portion of the circumference of third central lumen 448. Third central lumen 448 may be configured to receive a third tool and/or a third material.

FIG. 5B illustrates sheath 430 in a second configuration (e.g., an open, or expanded, configuration). As explained in more detail below, the portions of flexible portions 434A, 434B, 434C, 434D forming outer wall 430D′ of sheath 430 may differ in the second, expanded configuration. For example, when a tool 452 having a diameter larger than the second central lumen 440 is inserted through second central lumen 440, arms (e.g., first arm 436A, second arm 436B, third arm 436C, and fourth arm 436D may break, or split, at their corresponding points 437, 443, and 445. As such, a diameter of sheath 430 may increase from the first configuration to the second configuration. In the second configuration, third central lumen 448 may merge, or combine, with second central lumen 440. Second wall portion 435B of first flexible portion 434A may unfold, such that, for example, first central lumen 438 is eliminated. In aspects, first tool 450 may be released from first central lumen 438 in the second configuration. First tool 450 may be released radially (e.g., from a radial side of sheath 430).

In the expanded configuration (FIG. 5B), a circumference/perimeter of second central lumen 440 may be defined by each rigid portion 432A, 432B, 432C, 432D and each flexible portion 434A, 434B, 434C, 434D. In these aspects, outer wall 430D′ in the expanded configuration may include each rigid portion 432A, 432B, 432C, 432D and each wall portion 435A, 435B, 435C of each flexible portion 434A, 434B, 434C, 434D.

Still referring to FIGS. 5A and 5B, one or more ancillary lumens 442 may extend through each rigid portion 432A, 432B, 432C, 432D. Ancillary lumen(s) 442 may have any or all of the same characteristics of ancillary lumen(s) 142, 242, and/or 342, discussed above. In further aspects, ancillary lumens 442 may be omitted from one or more of rigid portions 432A, 432B, 432C, and/or 432D. Omitting ancillary lumen(s) 442 may allow for a larger cross-sectional area of second central lumen 440 (e.g., in the second configuration) and/or third central lumen 448 (e.g., in the first configuration). As shown in FIGS. 5A and 5B, each rigid portion may include two ancillary lumens 442, although such a number is merely exemplary. Ancillary lumens of a given rigid portion 432A, 432B, 432C, 432D may have different sizes, such that rigid portion 432A, 432B, 432C, 432D is asymmetrical. Alternatively, ancillary lumens 442 may have the same size.

In some aspects, a material may be delivered via any of the lumens of sheath 430 (e.g., via first central lumen 438, second central lumen 440, and/or peripheral lumens 442). For example, a first material may be delivered via first central lumen 438. A second material may be delivered via second central lumen 440. Together or separately, each of the first material and second material may be injected through their respective lumens of sheath 430. In aspects, a third material may be delivered via the one or more peripheral lumen(s) 442.

In some examples, a fourth material may be disposed within third central lumen 448, for example, when sheath 430 is in the first configuration. Accordingly, when sheath 430 transitions to the second configuration, the fourth material disposed within third central lumen 448 may combine and/or cross-link with the second material disposed within second central lumen 440, for example, prior to being delivered to the target site.

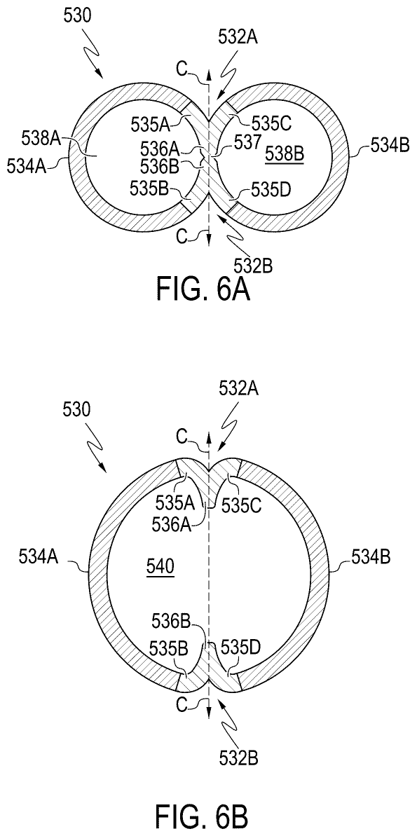

FIGS. 6A and 6B illustrate cross-sectional views of an exemplary sheath 530 in a first configuration (FIG. 6A) and a second configuration (FIG. 6B). Except as described below, sheath 530 may have any or all of the same characteristics of sheaths 130, 230, 330, and/or 430, described above. For example, sheath 530 may include a plurality of rigid portions and a plurality of flexible portions. In particular, sheath 530 may include a first rigid portion 532A and a second rigid portion 532B. Sheath 530 may further include a first flexible portion 534A and a second flexible portion 534B. In some aspects, the rigid portions and the flexible portions of sheath 530 may be integrally formed or otherwise fixedly coupled to one another (e.g., overmolded or co-extruded).

Sheath 530 may also include a first lumen 538A and a second lumen 538B in the first configuration (FIG. 6A). In aspects, sheath 530 may be symmetric about a centerline “C” that extends perpendicularly to a longitudinal axis of sheath 530. The longitudinal axis of sheath 530 may extend into and out of the page of FIGS. 6A and 6B. In other aspects, sheath 530 may be asymmetric about centerline “C.” For example, first lumen 538A may be larger than second lumen 538B, or vice versa. Additionally or alternatively, first lumen 538A may have a different cross-sectional shape as compared to second lumen 538.

In the first configuration (FIG. 6A), a first portion of each of first rigid portion 532A, second rigid portion 532B, and first flexible portion 534A may at least partially define first lumen 538A. First lumen 538A may be defined on a first side of centerline “C” in the first configuration. For example, a first wall portion 535A and first arm 536A of first rigid portion 532A, a second wall portion 535B and second arm 536B of second rigid portion 532B, and first flexible portion 534A may together define first lumen 538A in the first configuration. First arm 536A and second arm 536B may abut one another, or be otherwise coupled, at point 537 in the first configuration.

First rigid portion 532A, second rigid portion 532B, and second flexible portion 534B may define second lumen 538B on a second, opposite side of centerline “C” in the first configuration. For example, a third wall portion 535C and first arm 536A of first rigid portion 532A, a fourth wall portion 535D and second arm 536B of second rigid portion 532B, and second flexible portion 534B may each at least partially define second lumen 538B in the first configuration (FIG. 6A).

Each of first lumen 538A and second lumen 538B may be configured to receive one or more tools and/or one or more materials. Sheath 530 may be configured to transition between the first configuration (FIG. 6A) and second configurations (FIG. 6B).

FIG. 6B illustrates sheath 530 in the second (e.g., open, or expanded) configuration. To transition sheath 530 from the first configuration (FIG. 6A) to the second configuration (FIG. 6B), a tool having a diameter larger than a diameter of the first lumen 538A and/or a diameter of the second lumen 538B may be inserted through at least one of first lumen 538A or second lumen 538B. For example, when the tool having a larger diameter is inserted through the first lumen 538A or the second lumen 538B, first arm 536A and second arm 536B may break, or separate, at point 537. As such, first lumen 538A may merge, or combine, with second lumen 538B, for example, to form a single, central lumen 540. In aspects, first flexible portion 534A and second flexible portion 534B may flex, or bend, to accommodate the tool and/or the formation of central lumen 540. In some aspects, first rigid portion 532A and second rigid portion 532B may also flex or bend to accommodate the formation of central lumen 540.

Additionally or alternatively, sheath 530 may transition from the first configuration (FIG. 6A) to the second configuration (FIG. 6B) by injecting a material through first lumen 538A and/or second lumen 538B. The material may apply pressure within the first lumen 538A and/or second lumen 538B, for example, to force first arm 536A and second arm 536B to break, or split, at point 537, thereby forming central lumen 540.

In some aspects, a part of a multi-part material may be contained within first lumen 538A and second lumen 538B, respectively. Accordingly, when sheath 530 transitions to the second configuration (FIG. 6B), the parts of the multi-part material may come in contact with one another and a reaction may or may not occur, for example, prior to being delivered to the target site. In some aspects, the materials may combine and/or cross-link with one another.

Central lumen 540 may have a larger cross-section as compared to each of first lumen 538A and second lumen 538B. In aspects, central lumen 540 may be configured to receive additional tools and/or additional materials (e.g., when sheath 530 is in the second configuration). Sheath 530 may transition from the second configuration (FIG. 6B) to the first configuration (FIG. 6A) by, for example, removing the tool(s) and/or material(s) from central lumen 540. First flexible portion 534A and second flexible portion 534B may flex so as to accommodate a size of a tool or a volume of fluid extended through lumen 540. For example, in the second configuration, first flexible portion 534A and second flexible portion 534B may have alternative shapes to that shown in FIG. 6B. For example, first flexible portion 534A and second flexible portion 534B may flex less radially outward, giving sheath 530 an oblong shape with a longer axis along centerline C. Alternatively, first flexible portion 534A and second flexible portion 534B may flex more radially outward, giving sheath 530 an oblong shape with a longer axis perpendicular to centerline C. In some examples, first flexible portion 534A and second flexible portion 534B may stretch or flex asymmetrically.

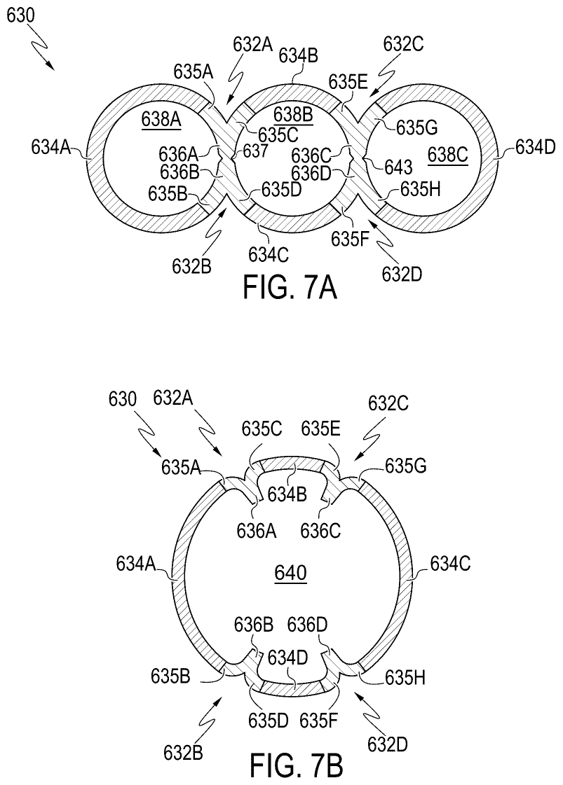

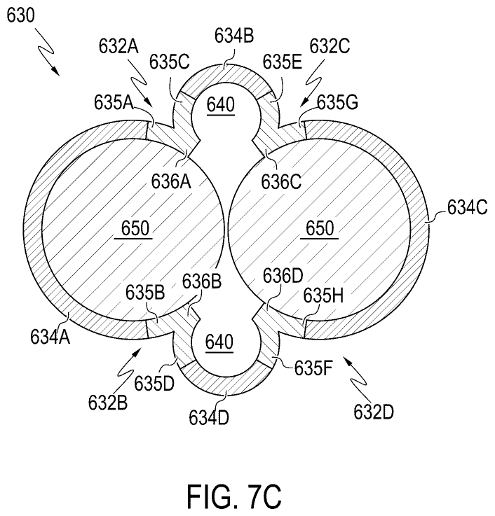

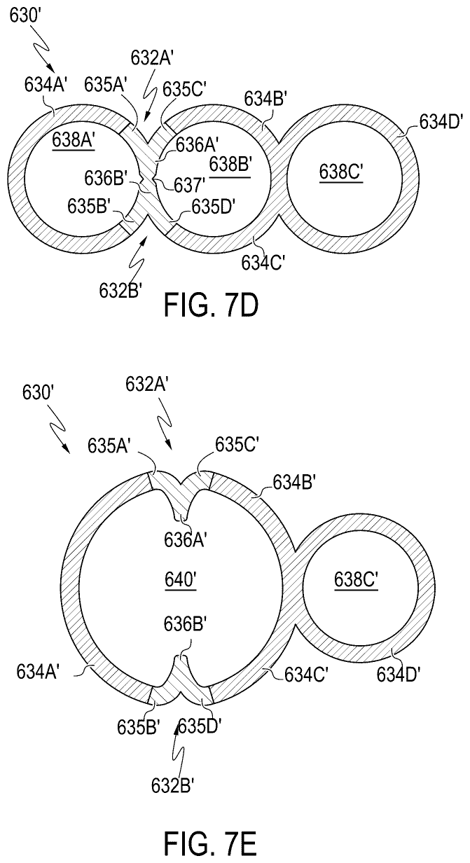

FIGS. 7A-7C illustrate cross-sectional views of an exemplary sheath 630 in a first configuration (FIG. 7A), a second configuration (FIG. 7B), and a third configuration (FIG. 7C). For example, FIG. 7B illustrates sheath 630 in the second configuration without a tool disposed within a central lumen of sheath 630, and FIG. 7C illustrates sheath 630 in the third configuration with at least one tool 650 disposed within the central lumen of sheath 630. Except as described below, sheath 630 may have any or all of the same characteristics of sheaths 130, 230, 330, 430, and/or 530, described above. For example, sheath 630 may include a plurality of rigid portions and a plurality of flexible portions. In particular, sheath 630 may include a first rigid portion 632A, a second rigid portion 632B, a third rigid portion 632C, and a fourth rigid portion 632D. Sheath 630 may further include a first flexible portion 634A, a second flexible portion 634B, a third flexible portion 634C, and a fourth flexible portion 634D. Sheath 630 may include a first lumen 638A, a second lumen 638B, and a third lumen 638C in the first configuration (FIG. 7A). In some aspects, the rigid portions and the flexible portions of sheath 630 may be integrally formed or otherwise fixedly coupled to one another (e.g., overmolded or co-extruded).

Referring primarily to FIG. 7A, second lumen 638B may be disposed between first lumen 638A and third lumen 638C. For example, a straight line may be drawn through center points of each of first lumen 638A, second lumen 638B, and third lumen 638C. Although first lumen 638A, second lumen 638B, and third lumen 638C are shown having a same size and/or shape in the first configuration in FIG. 7A, one or more of first lumen 638A, second lumen 638B, and/or third lumen 638C may have a different size and/or shape. For example, second lumen 638B may have a larger diameter than first lumen 638A and/or third lumen 638C. In some aspects, second lumen 638B may be ovular, and one or both of first lumen 638A and third lumen 638C may be circular, or vice versa. Many other combinations and configurations are contemplated. In some aspects, sheath 630 may include additional flexible portions and rigid portions, for example, to accommodate additional lumens.

In the first configuration of sheath 630, first lumen 638A may be defined similarly to first lumen 538A of sheath 530 of FIG. 6A. For example, a first portion of each of first rigid portion 632A, second rigid portion 632B, and first flexible portion 634A may define first lumen 638A in the first configuration (e.g., with a first arm 636A of first rigid portion 632A and a second arm 636B of second rigid portion 632B abutting one another, or otherwise coupled, at point 637.) In particular, a first wall portion 635A and first arm 636A of first rigid portion 632A, a second wall portion 635B and second arm 636B of second rigid portion 632B, and first flexible portion 634A may together define first lumen 638A in the first configuration.

First rigid portion 632A, second rigid portion 632B, second flexible portion 534B, and third flexible portion 634C may at least partially define second lumen 638B in the first configuration (e.g., with a first arm 636A and a second arm 636B abutting one another, or otherwise coupled, at a first point 637, and with a third arm 636C of third rigid portion 632C, and a fourth arm 636D of fourth rigid portion 632D abutting one another, or otherwise coupled, at a second point 643). In particular, in the first configuration, second lumen 638B may be defined by a third wall portion 635C and first arm 636A of first rigid portion 632A, a fourth wall portion 635D and second arm 636B of second rigid portion 632B, second flexible portion 634B, third flexible portion 634C, a fifth wall portion 635E and third arm 636C of third rigid portion 632C, and a sixth wall portion 635F and fourth arm 636D of fourth rigid portion 632C.

Still referring primarily to FIG. 7A, third lumen 638C may also be defined in the first configuration (e.g., with third arm 636C and fourth arm 636D abutting one another, or otherwise coupled, at second point 643). Third lumen 638C may be defined by, for example, a seventh wall portion 635G and third arm 636C of third rigid portion 632C, fourth flexible portion 634D, and an eighth wall portion 635H and fourth arm 636D of fourth rigid portion 632D.

In aspects, one or more of first lumen 638A, second lumen 638B, and third lumen 638C may be configured to receive one or more tools (e.g., one or more of tool 152, 252, 352, 452 of the previous figures) and/or one or more materials. In some aspects, receiving the one or more tools and/or the one or more materials may transition sheath 630 from the first configuration (FIG. 7A) to the second configuration (FIG. 7B) and/or the third configuration (FIG. 7C).

FIG. 7B illustrates sheath 630 in the second (e.g., open, or expanded) configuration. To transition sheath 630 from the first configuration (FIG. 7A) to the second configuration (FIG. 7B), a tool having a diameter larger than a diameter of first lumen 638A, second lumen 638B, and/or third lumen 638C may be inserted through the respective lumen (e.g., at least one of the first lumen 638A, the second lumen 638B, and/or the third lumen 638C). For example, when the tool having a larger diameter is inserted through second lumen 638B, first arm 636A and second arm 636B may break, or separate, at first point 637. Additionally, third arm 636C and fourth arm 636D may break, or separate, at second point 643. As such, first lumen 638A, second lumen 638B, and third lumen 638C may merge, or combine, for example, to form a single, central lumen 640. One or more flexible portions may flex, or bend, to accommodate the tool and/or to otherwise accommodate the formation of central lumen 640. For example, one or more of first flexible portion 634A, second flexible portion 634B, third flexible portion 634C, and/or fourth flexible portion 634D may flex or bend upon receiving the tool.

Additionally or alternatively, sheath 630 may transition from the first configuration (FIG. 7A) to the second configuration (FIG. 7B) by injecting a material through second lumen 638B. The material may apply pressure within second lumen 638B, such that, for example, first arm 636A and second arm 636B break, or separate, at first point 637, and third arm 636C and fourth arm 636D may break, or separate, at second point 643. In these aspects, central lumen 640 of sheath 630 may be formed.

Central lumen 640 may have a larger cross-section as compared to each of first lumen 638A, second lumen 638B, and/or third lumen 638C. In aspects, central lumen 640 may be configured to receive additional tools and/or additional materials (e.g., when sheath 630 is in the second configuration). Sheath 630 may transition from the second configuration (FIG. 6B) to the first configuration (FIG. 6A) by, for example, removing the tool(s) and/or material(s) from central lumen 640. In some aspects, the flexible portions may be configured to stretch, for example, to accommodate larger tool(s) and/or additional material(s) through central lumen 640. In aspects, an overall cross-sectional shape of sheath 630 may be elongated, or ovular, in the first configuration. In the second configuration, a cross-sectional shape of sheath 630 may be circular, or round.

FIG. 7C illustrates sheath 630 in the third configuration. The third configuration is similar to the second configuration (FIG. 7B). As discussed above, sheath 630 may be configured to receive one or more tools or materials. FIG. 7C illustrates sheath 630 having two tools 650 disposed within lumen 640 in the third configuration. A cross-sectional shape of sheath 630 in the third configuration may resemble a four-leaf clover, or a modified quatrefoil. For example, the cross-sectional shape of sheath 630 may include at least four lobes arranged approximately 90 degrees from one another. Two opposing lobes of the at least four lobes may have a same or similar largest diameter in the third configuration. For example, in the third configuration, a first diameter of sheath 630 may be less than a second diameter that is measured approximately 90 degrees relative to the first diameter. In these aspects, the cross-sectional shape of sheath 630 may be elongated. A width/diameter of tools 650 may be such that lumen 640 is opened more in one direction (e.g., a left/right direction shown in FIG. 7C) than in another (e.g., opposite) direction (e.g., an up/down direction of FIG. 7C).

In the third configuration, first flexible portion 634A, first wall portion 635A, first arm 636A, second arm 636B, and second wall portion 635B may at least partially surround a circumference of one of tools 650 inserted through central lumen 640. Similarly, third arm 636C, seventh wall portion 635G, third flexible portion 634C, eighth wall portion 635H, and fourth arm 636D may at least partially surround a circumference of a second tool 650 inserted through central lumen 640. In these aspects, tools 650 may be supported, for example, when tools 650 are disposed within lumen 640. In some aspects, first arm 636A, second arm 636B, third arm 636C, and fourth arm 636D may extend at least partially into lumen 640 in the third configuration.

Although not shown, in a third configuration of sheath 630, exactly two lumens may be defined. For example, in the third configuration, first arm 636A and second arm 636B may be separated or disconnected at first point 637. In these aspects, first lumen 638A and second lumen 638B may merge, thereby forming a single lumen. Third lumen 638C may remain the same as in the first configuration. In these aspects, exactly two lumens may be defined in the third configuration. The exactly two lumens may be defined, for example, by inserting at least one tool having a diameter greater than first lumen 638A through first lumen 638A and/or injecting at least one material into first lumen 638A.

In other aspects, third arm 636C and fourth arm 636D may be separated or disconnected at second point 643 such that second lumen 638B and third lumen 638C may merge to form a single lumen, and first lumen 638A may remain the same. In these aspects, exactly two lumens may again be defined in the third configuration. The exactly two lumens may be defined, for example, by inserting at least one tool having a diameter greater than third lumen 638C through third lumen 638C and/or injecting at least one material into third lumen 638C.

FIGS. 7D and 7E illustrate a sheath 630′ in a first configuration (FIG. 7D) and a second configuration (FIG. 7E). Sheath 630′ may have any or all of the same characteristics of sheath 630, discussed above with respect to FIGS. 7A-7C, unless specified below. Corresponding structures of sheath 630′ are indicated by adding a “prime” to reference numbers related to sheath 630. For example, sheath 630′ may be configured to define at least three lumens 638A′, 638B′, 638C′ in the first configuration (FIG. 7A). In the second configuration (FIG. 7B), exactly two lumens may be defined, as discussed below.

Sheath 630′ may include a plurality of rigid portions and a plurality of flexible portions. In particular, sheath 630′ may include a first rigid portion 632A′ and a second rigid portion 632B′ (e.g., exactly two rigid portions). Sheath 630 may further include a first flexible portion 634A′, a second flexible portion 634B′, and a third flexible portion 634D′. As compared with sheath 630, sheath 630′ may have fewer rigid portions and fewer discrete flexible portions. Second flexible portion 634B′ and third flexible portion 634D′ may be directly coupled to one another. For example, second flexible portion 634B′ may have a shape similar to first flexible portion 634A′ and/or 634A (e.g., a “C” shape), and third flexible portion 634D′ may have an approximately ring shape.

Referring to FIG. 7D, in the first configuration, a first lumen 638A′ may be at least partially defined by first flexible portion 634A′, a first wall portion 635A′ of first rigid portion 632A′, a first arm 636A′ of first rigid portion 632A′, a second arm 636B′ of second rigid portion 632B′, and a second wall portion 635B′ of second rigid portion 632B′. A second lumen 638B′ may be at least partially defined by second flexible portion 634B′, a third wall portion 635C′ of first rigid portion 632A′, a fourth wall portion 635D′ of second rigid portion 632B′, first arm 636A′, and second arm 636B′. In the second configuration, first arm 636A′ and second arm 636B′ may break apart, or separate, at point 637′ such that first lumen 638A′ and second lumen 638B′ may merge, thereby forming lumen 640′. In some aspects, first arm 636A′ and/or second arm 636B′ may protrude radially inward, for example, into lumen 640′. First arm 636A′ and/or second arm 636B′ may assist in providing support for an instrument (not shown) inserted through lumen 640′.

Sheath 630′ may transition from the first configuration (FIG. 7D) to the second configuration (FIG. 7E), for example, as a result of the insertion of one or more tools and/or materials though one or both of lumens 638A′ and 638B′.

Referring still to FIGS. 7D and 7E, third lumen 638C′ may be only defined by third flexible portion 634D′ in the first configuration (FIG. 7D) and in the second configuration (FIG. 7E). For example, third lumen 638C′ may remain intact between the first configuration (FIG. 7D) and the second configuration (FIG. 7E). Third lumen 638C′ may be configured to receive one or more tools and/or materials. In some examples, third flexible portion 634D′ and second flexible portion 634B′ may be integrally formed with one another and may share a wall that divides second lumen 638B′ from third lumen 638C′.

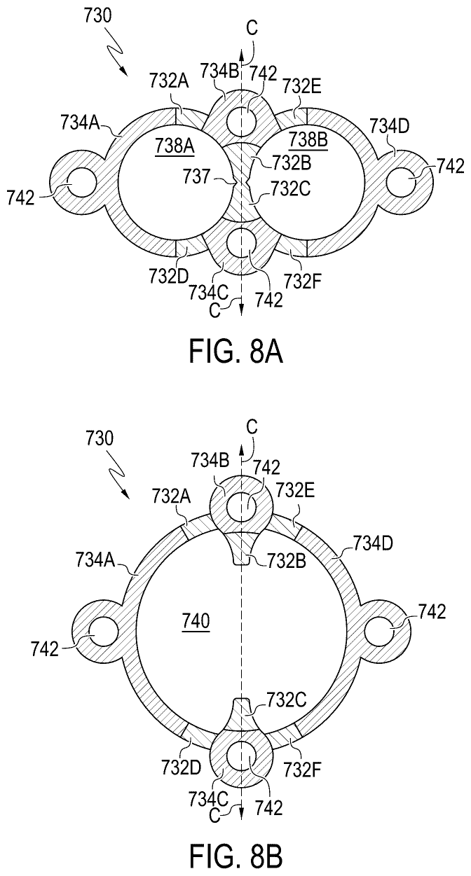

FIGS. 8A and 8B illustrate cross-sectional views of an exemplary sheath 730 in a first configuration (FIG. 8A) and a second configuration (FIG. 8B). Except as described below, sheath 730 may have any or all of the same characteristics of sheaths 130, 230, 330, 430, 530, and/or 630, described above. For example, sheath 730 may include a plurality of rigid portions and a plurality of flexible portions. In particular, sheath 730 may include a first rigid portion 732A, a second rigid portion 732B, a third rigid portion 732C, a fourth rigid portion 732D, a fifth rigid portion 732E, and a sixth rigid portion 732F. Sheath 730 may include a first flexible portion 734A, a second flexible portion 734B, a third flexible portion 734C, and a fourth flexible portion 734D. In some aspects, the rigid portions and the flexible portions of sheath 730 may be integrally formed or otherwise fixedly coupled to one another (e.g., overmolded or co-extruded). Second flexible portion 734B may be between first rigid portion 732A and fifth rigid portion 732E. Third flexible portion 734C may be between fourth rigid portion 732D and sixth rigid portion 732F.

Sheath 730 may further include a plurality of lumens. For example, sheath 730 may include a first lumen 738A and a second lumen 738B in the first configuration (FIG. 7A). Additional ancillary lumens may be defined by the flexible portions. For example, one or more ancillary lumens 742 may extend through each of first flexible portion 734A, second flexible portion 734B, third flexible portion 734C, and fourth flexible portion 734D. Ancillary lumen(s) 742 may have any or all of the same characteristics of ancillary lumen(s) 142, 242, 342, and/or 442, discussed above.

In aspects, sheath 730 may be symmetric about a centerline “C” that may extend perpendicularly to a longitudinal axis of sheath 730. In other aspects, sheath 730 may be asymmetric about centerline “C.” For example, first lumen 738A may be larger than second lumen 738B, or vice versa. Similarly, one or more ancillary lumens 742 may be differently sized and/or shaped.

In aspects, ancillary lumen(s) 742 may be arranged radially about a longitudinal axis of sheath 730. The longitudinal axis of sheath 730 may extend into and out of the page of FIGS. 8A and 8B. In aspects, one or more ancillary lumens 742 may be at least partially disposed between first lumen 738A and second lumen 738B in the first configuration. For example, one or more ancillary lumens 742 may extend across centerline “C.” In some aspects, ancillary lumen(s) 742 may extend radially outward relative to the longitudinal axis of sheath 730. In other aspects, ancillary lumen(s) 742 may be disposed within first lumen 738A and/or second lumen 738B.

Except as described below, first lumen 738A and second lumen 738B may be defined similarly as first lumen 538A and second lumen 538B of sheath 530 of FIG. 6A. In these aspects, first lumen 738A and second lumen 738B may have any or all of the same characteristics of first lumen 538A and second lumen 538B of sheath 530. For example, first lumen 738A may be at least partially defined by first flexible portion 734A, first rigid portion 732A, second flexible portion 734B, second rigid portion 732B, third rigid portion 732C, third flexible portion 734C, and fourth rigid portion 732D. Second lumen 738B may be at least partially defined by fourth flexible portion 734D, fifth rigid portion 732E, second flexible portion 734B, second rigid portion 732B, of third rigid portion 732C, third flexible portion 734C, and sixth rigid portion 732F.

In the first configuration (FIG. 8A), second rigid portion 732B may touch, or abut, third rigid portion 732C at a point 737. In the second configuration (FIG. 8B), second rigid portion 732B and third rigid portion 732C may break, or separate, at point 737. In these aspects, first lumen 738A and second lumen 738B may merge to form a single central lumen 740. One or more of the flexible portions and/or one or more of the rigid portions may bend or flex, for example, to accommodate the creation of central lumen 740. In the first configuration, sheath 730 may have a substantially oval, or elongated, shape. In the second configuration, sheath 730 may have a substantially circular shape. Ancillary lumen(s) 742 may remain unchanged from the first configuration to the second configuration.

Sheath 730 may be configured to transition from the first configuration (FIG. 8A) to the second configuration (FIG. 8B) and vice versa. For example, to transition sheath 730 from the first configuration (FIG. 7A) to the second configuration (FIG. 7B), a tool having a diameter larger than a diameter of the first lumen 738A and/or a diameter of the second lumen 738B may be inserted through the respective lumen. When the tool having a larger diameter is inserted through the first lumen 738A and/or the second lumen 738B, second rigid portion 732B and third rigid portion 732C may break, or separate, at point 737. As such, first lumen 738A may merge, or combine, with second lumen 738B, for example, to form central lumen 740. In some aspects, the flexible portions may be configured to stretch, for example, to accommodate larger tools through central lumen 740.

Additionally or alternatively, sheath 730 may transition from the first configuration (FIG. 8A) to the second configuration (FIG. 8B) by injecting a material through first lumen 738A and/or second lumen 738B. The material may apply pressure within the first lumen 738A and/or second lumen 738B, for example, to force second rigid portion 732B and third rigid portion 732Cto break, or split, at point 737, thereby forming central lumen 740. In some aspects, the flexible portions may be configured to stretch, for example, to accommodate additional materials through central lumen 740.

In aspects, first flexible portion 734A, second flexible portion 734B, third flexible portion 734C, and/or fourth flexible portion 734D may flex so as to accommodate a size of a tool or a volume of fluid extended through first lumen 738A and/or second lumen 738B. For example, in the second configuration (FIG. 8B), first flexible portion 734A, second flexible portion 734B, third flexible portion 734C, and/or fourth flexible portion 734D may have alternative shapes to that shown in FIG. 8B. For example, one or more of first flexible portion 734A, second flexible portion 734B, third flexible portion 734C, and/or fourth flexible portion 734D may flex less radially outward, giving sheath 730 an oblong shape with a longer axis along centerline C. Alternatively, first flexible portion 734A, second flexible portion 734B, third flexible portion 734C, and/or fourth flexible portion 734D may flex more radially outward, giving sheath 730 an oblong shape with a longer axis perpendicular to centerline C. In some examples, first flexible portion 734A, second flexible portion 734B, third flexible portion 734C, and/or fourth flexible portion 734D may stretch or flex asymmetrically.

Central lumen 740 may have a larger cross-section as compared to each of first lumen 738A and second lumen 738B. In aspects, central lumen 740 may be configured to receive additional tools and/or additional materials (e.g., when sheath 730 is in the second configuration). Sheath 730 may transition from the second configuration (FIG. 8B) to the first configuration (FIG. 8A) by, for example, removing the tool(s) and/or material(s) from central lumen 740. In the second configuration, second rigid portion 732B and third rigid portion 732C may protrude, or extend radially into, lumen 740. In these aspects, second rigid portion 732B and third rigid portion 732C may support a tool extending through lumen 740. For example, second rigid portion 732B and/or third rigid portion 732C may contact a tool or other accessory. In some aspects, second rigid portion 732B and/or third rigid portion 732C may retain a tool so that it is approximately coaxial with a central longitudinal axis of lumen 740.

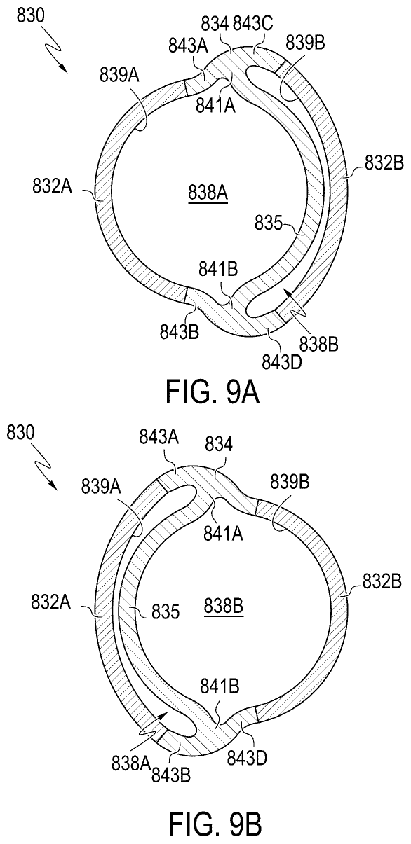

FIGS. 9A and 9B illustrate cross-sectional views of an exemplary sheath 830 in a first configuration (FIG. 9A) and a second configuration (FIG. 9B). Sheath 830 may have any or all of the same characteristics of sheath 130, 230, 330, 430, 530, 630, and/or 730 of the previous figures. For example, sheath 830 may include a first rigid portion 832A, a flexible portion 834, and a second rigid portion 832B. In particular, first rigid portion 832A and second rigid portion 832B may have any or all of the same characteristics of one or both of rigid portions 132A, 132B of FIGS. 2A and 2B. Flexible portion 834 may have any or all of the same characteristics of any of the flexible portions previously described with reference to FIGS. 2A-8B. Flexible portion 834 may be between first rigid portion 832A and second rigid portion 832B. In some aspects, first rigid portion 832A, flexible portion 834, and second rigid portion 832B may be integrally formed or otherwise fixedly coupled to one another (e.g., overmolded or co-extruded).

Flexible portion 834 may include a wall portion 835 extending between a first base 841A and a second base 841B. In aspects, wall portion 835 may at least partially define a first lumen 838A and a second lumen 838B. For example, first lumen 838A may be defined by a first inner surface 839A of first rigid portion 832A and wall portion 835. First inner surface 839A of first rigid portion 832A and wall portion 835 of flexible portion 834 may together define an entire circumference of first lumen 838A. Second lumen 838B may be defined by a second inner surface 839B of second rigid portion 832B and wall portion 835. For example, second inner surface 839B of second rigid portion 832B and wall portion 835 of second rigid portion 832B may together define an entire circumference of second lumen 838B.

In the first configuration (FIG. 9A), first lumen 838A may be in an expanded position and second lumen 838B may be in a collapsed position. For example, a portion of wall portion 835 of flexible portion 834 may abut or be adjacent to second inner surface 839B of second rigid portion 834B. In aspects, wall portion 835 may be rounded towards second inner surface 839B in the first configuration. Wall portion 835 may define a concave surface of first lumen 838A and a convex surface of second lumen 838B. In these aspects, first lumen 838A may have a first cross-sectional area, or a first volume, in the first configuration. Similarly, second lumen 838B may have a second cross-sectional area, or a second volume, in the first configuration. In aspects, the first cross-sectional area of first lumen 838A may be greater than the second cross-sectional area of second lumen 838B in the first configuration.

In the second configuration (FIG. 9B), first lumen 838A may be in a collapsed position and second lumen 838B may be in an expanded position. For example, a portion of wall portion 835 of flexible portion 834 may abut or be adjacent to first inner surface 839A of first rigid portion 832A. In aspects, wall portion 835 may be rounded towards first inner surface 839A in the second configuration. Wall portion 835 may define a concave surface of second lumen 838B and a convex surface of first lumen 838A. In these aspects, first lumen 838A may have a third cross-sectional area, or a third volume, in the second configuration. Similarly, second lumen 838B may have a fourth cross-sectional area, or a fourth volume, in the second configuration. The third cross-sectional area of first lumen 838A may be smaller than the fourth cross-sectional area of second lumen 838B in the second configuration. Comparatively, a cross-sectional area of an entirety of sheath 830 may be the same in the first and second configurations.

To accommodate movement of wall portion 835 between the first and second configurations, first base 841A and second base 841B may be flexible and/or movable. For example, each of first base 841A and second base 841B may include two or more arms that are configured to bend and flex to assist in transitioning sheath 830 from the first configuration to the second configuration. For example, an inner surface of a first arm 843A of first base 841A and an inner surface of a second arm 843B of second base 841B may curve away from first lumen 838A, for example, to assist in forming first lumen 838A in the first configuration. An inner surface of a third arm 843C of first base 841A and an inner surface of a fourth arm 843D of second base 841B may curve into second lumen 838B, for example, to assist in forming second lumen 838B in the first configuration. In the second configuration, the inner surface of first arm 843A and the inners surface of second arm 843B may curve into first lumen 838Ato assist in forming first lumen 838A in the second configuration, and the inner surface of third arm 843C and the inner surface of fourth arm 843D may curve away from second lumen 838B to assist in forming second lumen 838B in the second configuration.

In the first configuration (FIG. 9A), first lumen 838A may be configured to receive one or more tools and/or one or more materials. For example, a first tool having a first diameter may be inserted through first lumen 838A and/or a first material having a first volume may be injected through first lumen 838A. The tool may have a diameter large enough to force, or move, wall portion 835 towards second inner surface 839B of second rigid portion 832B. Additionally or alternatively, a volume of the material injected through first lumen 838A may be sufficient to force, or move, wall portion 835 towards second inner surface 839B of second rigid portion 832B.

Second lumen 838B may be configured to also receive one or more tools and/or one or more materials in the first configuration. For example, a second tool having a second, smaller diameter may be inserted through second lumen 838B. Additionally or alternatively, a second material having a second, lesser volume may be injected through second lumen 838B in the first configuration. The second tool and/or second material may be small enough, for example, such that wall portion 835 is not forced, or moved, away from second inner surface 839B of second lumen 838B.

To transition sheath 830 to the second configuration (FIG. 9B), the first tool and/or first material may be at least partially removed from first lumen 838A. A third tool having a diameter greater than second lumen 838B (e.g., when sheath 830 is in the first configuration) may be inserted through second lumen 838B such that wall portion 835 is forced, or moved, towards first inner surface 839A of first rigid portion 832A. Additionally or alternatively, a third material having sufficient volume to force, or move, wall portion 835 towards first inner surface 839A of first rigid portion 832A may be injected into second lumen 838B. Accordingly, the diameter of second lumen 838B increases as the diameter of first lumen 838A decreases. Sheath 830 may be configured to similarly transition from the second configuration to the first configuration. In some aspects, sheath 830 may be configured to transition between the first and second configurations multiple times during a single use.

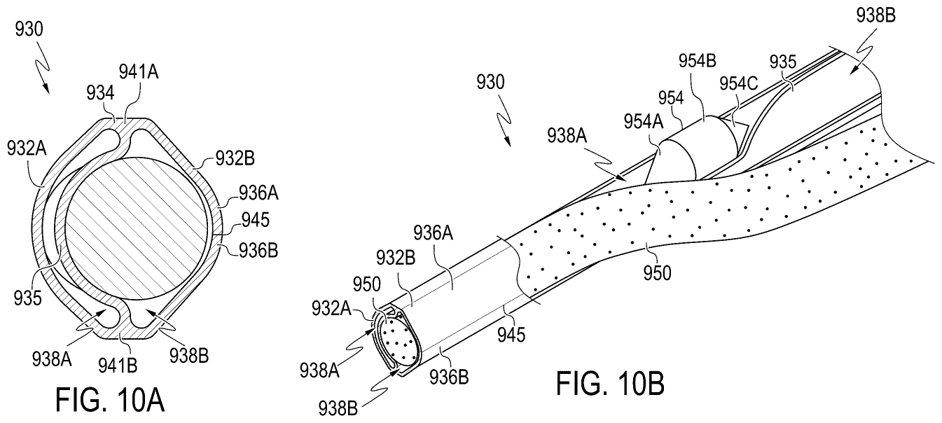

In some aspects, a wall defining an outer perimeter of first rigid portion 832A and/or second rigid portion 832B may include an opening for example, to assist with delivering the one or more tools and/or the one or more materials radially out of first lumen 838A and/or second lumen 838B. For example, FIGS. 10A and 10B illustrate a sheath 930 having at least one flexible portion with a seam 945 extending longitudinally along at least a portion of sheath 930.

Various aspects of sheath 830 may be configured to flex so as to accommodate a size of a tool or a volume of fluid extended through first lumen 838A and/or second lumen 838B. For example, flexible portion 834, first rigid portion 832A, second rigid portion 832B, and wall portion 835 may have alternative shapes to that shown in FIGS. 9A and 9B. In some examples, flexible portion 834, first rigid portion 832A, second rigid portion 832B, and/or wall portion 835 may stretch or flex asymmetrically. In other aspects, an entirety of sheath 830 may have a same or similar flexibility. For example, flexible portion 834, first rigid portion 832A, second rigid portion 832B, and wall portion 835 may each have the same or similar flexibility.

FIG. 10A illustrates a cross-sectional view of sheath 930, and FIG. 10B illustrates a perspective view of sheath 930. For example, FIG. 10B illustrates sheath 930 with a portion of second rigid portion 932B removed at a proximal portion of sheath 930, for example, to provide an internal view of sheath 930 as tool 950 is being ejected from second lumen 938B of sheath 930. In some aspects, FIG. 10A illustrates sheath 930 in a first configuration and FIG. 10B illustrates sheath 930 in a second configuration. In the second configuration, a tool 950 may be at least partially disposed within second lumen 938B. For example, in the second configuration, a proximal portion of tool 950 may be ejected radially from second lumen 938B, as will be described in further detail below. In some aspects, in the second configuration, a distal portion of tool 950 may be still be disposed within second lumen 938B.

Referring to both FIGS. 10A and 10B, sheath 930 may have any or all of all of the characteristics of sheath 130, 230, 330, 430, 530, 630, 730, 830, described above. In particular, sheath 930 may have any or all of the same characteristics as sheath 830, except as described below.

Sheath 930 may include a first lumen 938A and second lumen 938B. First lumen 938A may be configured to receive an ejecting tool 954, described below. Second lumen 938B may be configured to receive a tool 950 and/or a material. Tool 950 may have any or all of the tools described with reference to the previous figures. Sheath 930 may include a first rigid portion 932A, a flexible portion 934, and a second rigid portion 932B. Flexible portion 934 may be between first rigid portion 932A and second rigid portion 932B. An entire circumference of first lumen 938A may be defined by first rigid portion 932A and a wall portion 935 of flexible portion 934. An entire circumference of second lumen 938B may be defined by a second rigid portion 932B and wall portion 935 of flexible portion 934.

Second rigid portion 932B may include a first arm 936A and a second arm 936B. First arm 936A may extend from a first base 941A. Second arm 936B may extend from a second base 941B. In some aspects, a seam 945 may be formed between the first end (e.g., an end opposite first base 941A) of first arm 936A and the second end (e.g., a free end opposite second base 941B) of second arm 936B in the first configuration. In some aspects, the first end of first arm 936A and the second end of second arm 936B may abut, but not overlap, in the first configuration. In other aspects, a first end of first arm 936A may overlap with a second end of second arm 936B in the first configuration.

As shown in FIG. 10B, seam 945 may extend along at least a portion of a longitudinal length of sheath 930. In aspects, seam 945 may include one or more perforations, notches, slits, or other surface features to assist with the separation of first arm 936A and second arm 936B. For example, in a first configuration, first arm 936A and second arm 936B may be joined at seam 945, such that second lumen 938B is closed. In some aspects, seam 945 may form a liquid-tight seal, for example, to prevent tools or materials from escaping second lumen 938B in the first configuration.

To eject tool 950 from second lumen 938B, ejecting tool 954 may be inserted through first lumen 938A. Ejecting tool 954 may be cylindrically shaped. In some aspects, ejecting tool 954 may have a tapered distal portion 954A, a cylindrical central portion 954B, and/or a tapered proximal portion 954C. A position of ejecting tool 954 within first lumen 938A may be controlled, for example, using a fluid and/or a control element. For example, a fluid (e.g., a gas or a liquid) may be injected into first lumen 938A, proximal of ejecting tool 954. The fluid may propel ejecting tool 954 distally. In other aspects, a control element (e.g., a wire, cable, etc.) may be fixed to ejecting tool 954. A user may thereby control a position of ejecting tool 954 within first lumen 938A. For example, the user may push or extend the control element distally, thereby moving ejecting tool 954 distally. Similarly, the user may pull or retract the control element proximally, thereby moving ejecting tool 954 proximally. In some aspects, a bolus of fluid or gas may be used in lieu of ejecting tool 954. For example, the bolus may be injected through first lumen 938A and function similar to ejecting tool 954, as described in further detail below.

Ejecting tool 954 may be configured to eject tool 950 from second lumen 938B (e.g., radially outward, or away from a central longitudinal axis of sheath 930). For example, as ejecting tool 954 translates distally within first lumen 938A, ejecting tool 954 may move or force wall portion 935 towards second rigid portion 932B. As wall portion 935 move towards second rigid portion 932B, wall portion may push tool 950 against first arm 936A and second arm 936B of second rigid portion 932B such that first arm 936A and second arm 936B separate at seam 945 and tool 950 is ejected radially outward from second lumen 938B.

Sheath 930 may be configured such that, for example, a portion of seam 945 that is distal of ejecting tool 954 remains intact. A portion of seam 945 that is adjacent to or proximal of ejecting tool 954 may be broken. In some aspects, seam 945 may reform, for example, proximal of ejecting tool 954. Seam 945 may be configured to reform proximally of ejecting tool 954, for example, without tool 950 disposed within second lumen 938B. In these aspects, multiple tools and/or materials may be ejected radially outward from second lumen 938B.

In some aspects, a softness or firmness of sheath 930 may vary. For example, first rigid portion 932A may be firmer as compared to second rigid portion 932B. Similarly, wall portion 935 may be softer than first rigid portion 932A and/or second rigid portion 932B. Additionally or alternatively, a softness or firmness of sheath 930 may vary along a longitudinal length. For example, proximal portions of sheath 930 may be softer as compared to distal portions of sheath 930, or vice versa.

Various aspects of sheath 930 may be configured to flex so as to accommodate a size of a tool or a volume of fluid extended through first lumen 938A and/or second lumen 938B. For example, flexible portion 934, first rigid portion 932A, second rigid portion 932B, and wall portion 935 may have alternative shapes to that shown in FIGS. 10A and 10B. In some examples, flexible portion 934, first rigid portion 932A, second rigid portion 932B, and/or wall portion 8935 may stretch or flex asymmetrically.

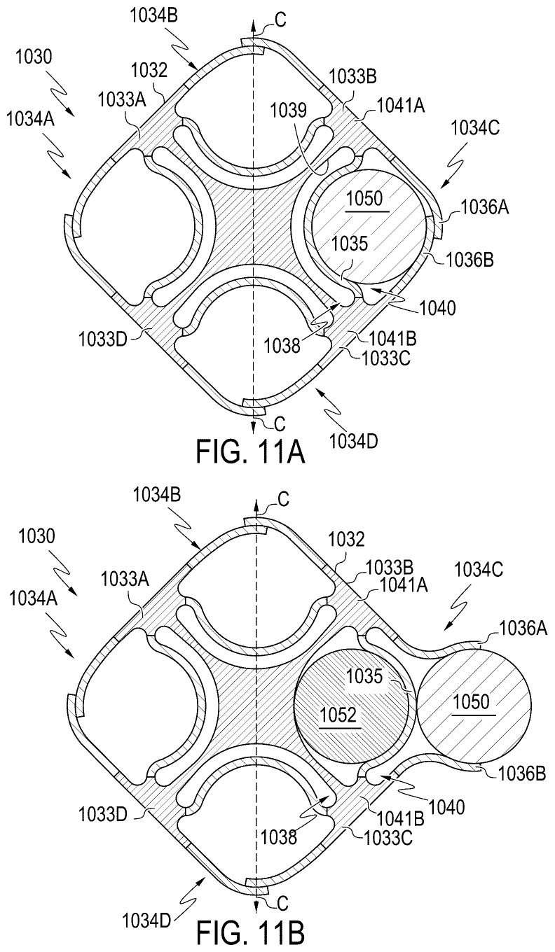

FIGS. 11A and 11B illustrate cross-sectional views of an exemplary sheath 1030 in a first configuration (FIG. 11A) and a second configuration (FIG. 11B). Sheath 1030 may have any or all of the same characteristics of sheath 130, 230, 330, 430, 530, 630, 730, 830, and/or 930. Sheath 1030 provides an exemplary configuration that combines aspects of two or more sheaths described herein. In particular, sheath 1030 includes aspects of sheath 230 of FIGS. 3A and 3B, and aspects of sheath 330 of FIG. 4, although sheath 1030 may include aspects of sheath 130, 430, 530, 630, 730, 830, and/or 930.

In particular, sheath 1030 may include a rigid portion 1032 that has an “X” or cross shape with a plurality of arms extending radially outward from a longitudinal axis of sheath 1030. The longitudinal axis of sheath 1030 may extend into and out of the page. Rigid portion 1032 may include a first arm 1033A, a second arm 1033B, a third arm 1033C, and a fourth arm 1033D. Sheath 1030 may include fewer arms or additional arms. In aspects, flexible portions 1034A, 1034B, 1034C, 1034D may extend between two arms of rigid portion 1032. Flexible portions 1034A, 1034B, 1034C, 1034D may have any or all of the characteristics of flexible portion 234 of sheath 230 of FIGS. 3A and 3B.

In aspects, for example, in a fully collapsed or a fully expanded configuration, sheath 1030 may be symmetric about a centerline “C” that may extend perpendicularly relative to the longitudinal axis of sheath 330. Features of sheath 1030 may be arranged, for example, in a radial pattern. In other aspects, sheath 1030 may be asymmetrical. For example, features of sheath 1030 on one side (e.g., the left or right side) of centerline “C” may not be on the other side (e.g., the right or left side) of centerline “C”, or vice versa.

In aspects, for example, in fully collapsed or fully contracted configurations, sheath 1030 may be symmetric about multiple centerlines. For example, sheath 1030 may be symmetric about two or four centerlines. In an example, sheath 1030 may be symmetric about centerlines that extend through (1) centers of flexible portions 1034B and 1034D (centerline “C”), (2) centers of flexible portions 1034A and 1034C, (3) centers of arms 1033A and 1033C, and/or (4) centers of arms 1033B and 1033D. For example, sheath 1030 may have an overall rounded square or rounded diamond shape.

Rigid portion 1032 may have any or all of the same characteristics of rigid portion 332 FIG. 4. Flexible portions 1034A, 1034B, 1034C, and/or 1034D may have any or all of the same characteristics of flexible portion 234 of FIGS. 3A and 3B. In some aspects, rigid portion 1032 may be integrally formed with or otherwise fixedly coupled to flexible portions 1034A, 1034B, 1034C, and/or 1034D.

Flexible portions 1034A, 1034B, 1034C, and 1034D may have any or all of the same characteristics as one another. For ease of description, third flexible portion 1034C is described in detail below. For example, third flexible portion 1034C is shown in the first configuration (FIG. 11A) and the second configuration (FIG. 11B). First flexible portion 1034A, second flexible portion 1034B and fourth flexible portion 1034D may have any or all of the same characteristics of third flexible portion 1034C.

Third flexible portion 1034C may be similarly shaped as flexible portion 234 of FIGS. 3A and 3B (and vice versa). For example, third flexible portion 1034C may include a wall portion 1035. Wall portion 1035 may extend between a first base 1041A and a second base 1041B of rigid portion 1032. In aspects, wall portion 1035 may at least partially define a first lumen 1038. First lumen 1038 may also be defined by an inner surface 1039 of rigid portion 1032. For example, inner surface 1039 of rigid portion 1032 and wall portion 1035 of flexible portion 1034 may together define an entire circumference of first lumen 1038. In the first configuration (FIG. 11A), first lumen 1038 may be in a collapsed position. For example, a portion of wall portion 1035 of flexible portion 1034C may abut or be adjacent to inner surface 1039. In aspects, wall portion 1035 may be rounded/bulge towards inner surface 1039. In these aspects, lumen 1038 may have a first cross-sectional area, or a first volume in the first configuration.

Third flexible portion 1034C may further include a first arm 1036A and a second arm 1036B. First arm 1036A may extend from first base 1041A. Second arm 1036B may extend from second base 1041B. In some aspects, a first end of first arm 1036A (an end opposite from an end coupled to first base 1041A) may overlap with a second end of second arm 1036B (an end opposite from an end coupled to second base 1041B) in the first configuration. In other aspects, first arm 1036A and second arm 1036B may not overlap. For example, a gap may be formed between the first end of first arm 1036A and the second end of second arm 1036B in the first configuration. Alternatively, the first end of first arm 1036A and the second end of second arm 1036B may abut, but not overlap, in the first configuration. In these aspects, a seam may be formed between first arm 1036A and second arm 136B (e.g., similar to seam 945 of FIGS. 10A and 10B).

In some aspects, wall portion 1035 may include arms that are similar to first arm 1036A and second arm 1036B. In other aspects, arms 1036A, 1036B may be replaced with features of sheath 130 (of FIGS. 2A and 2B). For example, third flexible portion 1034C may include a first wall portion (e.g., similar to first wall portion 135A of FIGS. 2A and 2B), a second wall portion (e.g., similar to second wall portion 135B of FIGS. 2A and 2B), and a third wall portion (e.g., similar to third wall portion 135C of FIGS. 2A and 2B) in addition to or in place of arms 1036A, 1036B of FIGS. 11A and 11B.

In the first configuration (FIG. 11A), first arm 1036A, second arm 1036B, and wall portion 1035 may together define a circumference/perimeter of a second lumen 1040. Second lumen 1040 may be adjacent to first lumen 1038, separated from one another by wall portion 1035.

Similar to sheath 230, sheath 1030 may be configured to eject one or more tools radially outward from second lumen 1040. For example, second lumen 1040 may be configured to receive a first tool 1050. First lumen 1038 may be configured to receive a second tool 1052. With second tool 1052 inserted through first lumen 1038, wall portion 1035 may be forced radially outward, such that, for example, wall portion 1035 is rounded away from inner surface 1039 and first tool 1050 is forced radially outward from first lumen 1038.

As first tool 1050 is forced radially outward, first arm 1036A and second arm 1036B may flex, or bend, outwards. First tool 1050 may be released from second central lumen 1040 via an opening formed between first arm 1036A and second arm 1036B. In these aspects, first tool 1050 may be released radially (e.g., from a radial side of sheath 1030).

Although not shown, rigid portion 1032 may include one or more ancillary lumens extending therethrough. The ancillary lumens may have any or all of the characteristics of ancillary lumens 142, 242, 342, and/or 742, described above.

In some aspects, a material may be delivered via any of the lumens of sheath 1030 (e.g., via first lumen 1038 and/or second lumen 1040). For example, a first material may be delivered via first lumen 1038. A second material may be delivered via second central lumen 1040. In some aspects, the second material may be configured to eject tool 1050 and/or a material disposed within the second lumen 1038B.

Various aspects of sheath 1030 may be configured to flex so as to accommodate a size of a tool or a volume of fluid extended through any one of the lumens of sheath 1030. For example, flexible portions 1034A, 1034B, 1034C, 1034D, rigid portion 1032, and wall portion 1035 may have alternative shapes to that shown in FIGS. 11A and 11B. In some examples, flexible portions 1034A, 1034B, 1034C, 1034D, rigid portion 1032, and wall portion 1035 may stretch or flex asymmetrically.

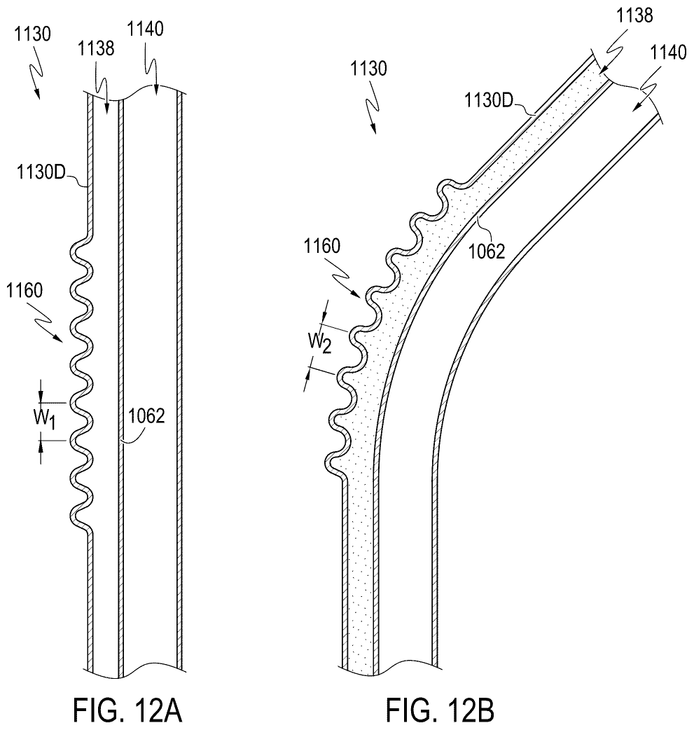

FIGS. 12A and 12B illustrate longitudinal cross-sectional views of an exemplary sheath 1130 in a first configuration (FIG. 12A) and a second configuration (FIG. 11B). Described in further detail below, sheath 1130 may be configured to bend in 2one or more directions, for example, via the injection of one or more fluids through a lumen of sheath 1130. Sheath 1130 may have any or all of the same characteristics of sheaths 130, 230, 330, 430, 530, 630, 730, 830, 930, and/or 1030. In aspects, each of the preceding embodiments may include one or more characteristics of sheath 1130.

Sheath 1130 may include a first lumen 1138 and a second lumen 1140 extending from a proximal end to a distal end of sheath 1130. Each of first lumen 1138 and second lumen 1140 may be defined by one or more walls of sheath 1130. For example, an outer wall 1130D of first lumen 1138 may include a plurality of ridges 1160 extending along at least a portion of a wall 1130D of sheath 1130.

In some aspects, wall 1130D may at least partially define an outer circumference of sheath 1130. For example, plurality of ridges 1160 may be disposed on an outer surface of sheath 1130. In other aspects, plurality of ridges 1160 may be disposed within an internal surface of sheath 1130. For example, plurality of ridges 1160 may be disposed along an inner wall 1162. Inner wall 1162 may be configured to separate one or more lumens from one another. In these aspects, plurality of ridges 1160 may not be visible when observing sheath 1130 externally.

Although a single portion of the plurality of ridges 1160 is illustrated in FIGS. 11A and 11B, sheath 1130 may include portions of the plurality of ridges 1160 elsewhere along a longitudinal length of sheath 1130. Additionally or alternatively, radial position of the plurality of ridges 1160 may vary along the length of sheath 1130. In these aspects, sheath 1130 may be configured to bend in one or more directions, for example, depending on a longitudinal and/or radial position of the plurality of ridges 1160 along sheath 1130.

First lumen 1138 may be configured to receive one or more fluids (e.g., gas (ses) or liquid(s)). Second lumen 1140 may be configured to receive a tool and/or a material. The one or more fluids is illustrated in stippling in FIG. 11B. Although not shown, first lumen 1138 may be capped, or closed, for example, to prevent the one or more fluids from being delivered from a distal end of first lumen 1138. In these aspects, the one or more fluids contained within first lumen 1138 may increase a pressure within first lumen 1138.

As the pressure increases within first lumen 1138, plurality of ridges 1160 may be configured to at least partially flatten. For example, in the first configuration (FIG. 11A), a gap between two ridges of the plurality of ridges 1160 may have a first width W1. In the second configuration (FIG. 11B), a gap between two ridges of the plurality of ridges 1160 may have a second width W2. First width W1 may be greater than second width W2. The width of each gap may increase, for example, to accommodate the pressure within first lumen 1138 created by the injection of the one or more fluids. A length of the discrete portion of plurality of ridges 1160 may thereby increase, for example, as the width of the gap between each ridge of the plurality of ridges 1160 increases (e.g., as plurality of ridges 1160 flattens).

Sheath 1130 may be configured to bend, for example, as the plurality of ridges 1160 are flattened/as spaces between the ridges 1160 increase in size. In some aspects, a length of wall 1130D (e.g., a distance between a proximal and distal end of wall 1130D) may increase, while a length of each of the remaining walls defining sheath 1130 remains constant. Sheath 1130 may be configured to bend relative to a longitudinal axis of sheath 1130 by approximately 1 degree to approximately 100 degrees or more. A bend angle of sheath 1130 may be controlled by, for example, an amount of fluid or pressure within first lumen 1138. For example, a higher pressure may result in a larger bend angle, while a smaller pressure may result in a smaller bend angle.

Sheath 1130 may be configured to straighten, for example, by decreasing the pressure within first lumen 1138. The pressure within first lumen 1138 may decrease, for example, by delivering the fluid contained within first lumen 1138. For example, a distal end of first lumen 1138 may be opened such that the fluid is ejected from first lumen 1128. In other aspects, the fluid may be removed from first lumen 1138 using negative pressure (e.g., a vacuum). In these aspects, sheath 1130 may be configured to bend, or articulate, via the injection of one or more fluids within first lumen 1138 of sheath 1130.

Any of features or characteristics of the preceding embodiments may be used in combination. While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a sheath having:
a rigid portion; and
a flexible portion;
wherein, in a first configuration, the sheath includes a first lumen and a second lumen, and wherein, in a second configuration, the sheath includes only the first lumen.

2. The medical device of claim 1, wherein the first lumen is defined by a wall of the rigid portion and a wall of the flexible portion.

3. The medical device of claim 1, wherein the flexible portion includes a first arm and a second arm, and wherein the second lumen is defined by a wall of the flexible portion, the first arm, and the second arm.

4. The medical device of claim 3, wherein, in the second configuration, the first arm and the second arm are configured to bend radially outward.

5. The medical device of claim 1, wherein the first lumen is configured to receive a first tool or a first material, and wherein the second lumen is configured to receive a second tool or a second material, wherein, upon insertion of the first tool or first material, the sheath transitions from the first configuration to the second configuration.

6. The medical device of claim 5, wherein, in the second configuration, the second tool or the second material is released from the second lumen.

7. The medical device of claim 1, wherein the flexible portion further includes a first wall portion, a second wall portion, and a third wall portion, wherein the first wall portion and the third wall portion are fixed to the rigid portion, and wherein the second wall portion extends between the first wall portion and the third wall portion.

8. The medical device of claim 7, wherein, in the first configuration, the second wall portion of the flexible portion is folded to define the second lumen, and wherein, in the second configuration, the second wall portion is unfolded to define a portion of an outer diameter of the sheath.

9. The medical device of claim 1, wherein the rigid portion is a first rigid portion, and the flexible portion is a first flexible portion, wherein the sheath includes a second rigid portion and a second flexible portion, and wherein, in the first and second configurations, the first rigid portion, the first flexible portion, the second rigid portion, and the second flexible portion define an outer perimeter of the sheath.

10. The medical device of claim 9, wherein a largest width of the sheath is smaller in the first configuration than in the second configuration.

11. The medical device of claim 9, wherein the sheath further includes a third lumen in the first configuration, and wherein, in the second configuration, the third lumen merges with the second lumen.

12. The medical device of claim 9, wherein the first flexible portion is between the first rigid portion and the second rigid portion, and wherein the second flexible portion is between the first rigid portion and the second rigid portion, opposite of the first rigid portion.

13. The medical device of claim 1, wherein, to transition the device from the first configuration to the second configuration, a tool, a material, or a fluid is inserted through the first lumen.

14. The medical device of claim 1, further comprising a handle at a proximal end of the sheath, wherein the handle is configured to provide fluid communication between a first port and the first lumen and a second port and the second lumen.

15. The medical device of claim 1, wherein the rigid portion defines a plurality of ancillary lumens.

16. A medical device, comprising:
a sheath having:
a rigid portion; and
a flexible portion fixed to the rigid portion, wherein the flexible portion includes:
a first wall portion;
a second wall portion; and
a third wall portion,
wherein, in a first configuration, the second wall portion is folded radially inward and defines a first lumen, and wherein the first wall portion, the third wall portion, and the rigid portion define a circumference of the sheath, and
wherein, in a second configuration, the second wall portion is unfolded, and the first wall portion, the second wall portion, the third wall portion, and the rigid portion define a circumference of the sheath.

17. The medical device of claim 16, wherein the sheath further includes a second lumen, wherein the second lumen is defined by the rigid portion and the flexible portion in the first and second configurations.

18. The medical device of claim 16, wherein the rigid portion defines a plurality of ancillary lumens.

19. The medical device of claim 16, wherein, a largest diameter of the sheath in the first configuration is less than a largest diameter of the sheath in the second configuration.

20. A medical device, comprising:
a sheath having:
a first rigid portion;
a second rigid portion;
a first flexible portion disposed between the first rigid portion and the second rigid portion, wherein the flexible portion includes:
a first wall portion;
a second wall portion; and
a third wall portion; and
a first lumen, wherein the first lumen is defined by the first rigid portion, the second rigid portion, and the first flexible portion, and
wherein, in a first configuration, the second wall portion is folded radially inward and defines a second lumen, and
wherein, in a second configuration, the second wall portion is unfolded, and the first wall portion, the second wall portion, the third wall portion, the first rigid portion and the second rigid portion define a circumference of the sheath.
